SEQUENCE LISTING (31 sequences)

SEQ ID NO: 1 - DNA, 23 bp, Artificial sequence
  Nicotiana benthamiana XylT gene oligonucleotide
gaggattatttagctcacccacg

SEQ ID NO: 2 - DNA, 22 bp, Artificial sequence
  Nicotiana benthamiana XylT gene oligonucleotide
agcagccaagactcctcaaaat

SEQ ID NO: 3 - DNA, 37 bp, Artificial sequence
  Nicotiana benthamiana XylT gene oligonucleotide
tatatgtcgactctagattagcaatgaagagcaagta

SEQ ID NO: 4 - DNA, 25 bp, Artificial sequence
  Nicotiana benthamiana XylT gene oligonucleotide
gctgtcaacgatacgctacgtaacg

SEQ ID NO: 5 - DNA, 23 bp, Artificial sequence
  Nicotiana benthamiana XylT gene oligonucleotide
ggtgctcatggagcaggtctaac

SEQ ID NO: 6 - DNA, 23 bp, Artificial sequence
  Nicotiana benthamiana XylT gene oligonucleotide
cgctacgtaacggcatgacagtg

SEQ ID NO: 7 - DNA, 29 bp, Artificial sequence
  Nicotiana benthamiana XylT gene oligonucleotide
agtcagagagagaagaagatgaacaagaa

SEQ ID NO: 8 - DNA, 24 bp, Artificial sequence
  Nicotiana benthamiana XylT gene oligonucleotide
gaactattcaaactgtcgagcgga

SEQ ID NO: 9 - DNA, 1551 bp, Nicotiana benthamiana
atgaacaagaaaaagctgaaaattcttgtttctctcttcgctctcaactcaatcactctctatctctacttctcttcccaccctgatcactctcgtcgcaaatccccccagaaccacttttcctcgtcggaaaaccaccatcataatttccactcttcaatcacttcccaatattccaggccttggcctattttgccctcctacctcccttggtctcaaaaccctaatgttgcttggagatcatgcgagggttacttcggtaatggttttactctcaaagttgatcttctcaaaacttcgccggagcttcaccggaaattcggcgaaaacaccgtcttcggagacggcggatggtttaggtgtttcttcagtgagactttgcagagttcgatctgcgagggaggcgcaatacgaatgaatccagacgagattttgatgtctcgtggaggtgagaaattggagtcggttattggtaggagtgaagatgatgaggtgcccgcgttcaaaactggagcttttcagattaaagttactgataaactgaaatttgggaaaaaattagtggatgaaaacttcttgaataaatacttaccggaaggtgcaatttcaaggcacactatgcgtgagttaatcgactctattcagttggttggcgccaatgattttcactgttctgagtggattgaggagccgtcacttttgattacacgatttgagtatgcaaaccttttccacacaattaccgattggtatagtgcatacgtggcatcgagggttactggcttgcccagtcggccacatttggtttttgtagatggccattgtgagacacaattggaggaaacatggaaagcacttttttcaagcctcacttatgctaagaactttagtggcccagtttgtttccgtcatgccgtcctctcgcctttgggatatgaaactgccctgtttaagggactgtcagaaactatagattgtaatggagcttctgctcatgatttgtggcaaaatcctgatgataagaaaactgcacggttatccgagtttggggagatgatcagggcagcctttggatttcctgttgatagacagaacatcccaaggacagtcacaggccctaatgtcctctttgttagacgtgaggattatttagctcacccacgtcatggtggaaaggtacagtctaggcttagcaatgaagagcaagtatttgattccataaagagctgggccttaaaccactcggagtgcaaattaaatgtaattagtggattgtttgcccacatgtccatgaaagagcaagttcgagcaatccaagatgcttctgtcattgttggtgctcatggagcaggtctaacccacatagtttctgcagcaccaaaagctgtaatactagaaattataagcagcgaatataggcgcccccattttgctctgattgctcaatggaaaggattggagtaccatcccatatatttggaggggtcttatgcggatcctccagtcgtgatcgacaagctcagcagcattttgaggagtcttgggtgctaa

SEQ ID NO: 10 - protein, 516 aa, Nicotiana benthamiana
MNKKKLKILVSLFALNSITLYLYFSSHPDHSRRKSPQNHFSSSENHHHNFHSSITSQYSRPWPILPSYLPWSQNPNVAWRSCEGYFGNGFTLKVDLLKTSPELHRKFGENTVFGDGGWFRCFFSETLQSSICEGGAIRMNPDEILMSRGGEKLESVIGRSEDDEVPAFKTGAFQIKVTDKLKFGKKLVDENFLNKYLPEGAISRHTMRELIDSIQLVGANDFHCSEWIEEPSLLITRFEYANLFHTITDWYSAYVASRVTGLPSRPHLVFVDGHCETQLEETWKALFSSLTYAKNFSGPVCFRHAVLSPLGYETALFKGLSETIDCNGASAHDLWQNPDDKKTARLSEFGEMIRAAFGFPVDRQNIPRTVTGPNVLFVRREDYLAHPRHGGKVQSRLSNEEQVFDSIKSWALNHSECKLNVISGLFAHMSMKEQVRAIQDASVIVGAHGAGLTHIVSAAPKAVILEIISSEYRRPHFALIAQWKGLEYHPIYLEGSYADPPVVIDKLSSILRSLGC

SEQ ID NO: 11 - DNA, 29 bp, Artificial sequence
  Nicotiana benthamiana XylT gene oligonucleotide
attctcgctctctcttcaaaaccgcaaat

SEQ ID NO: 12 - DNA, 29 bp, Artificial sequence
  Nicotiana benthamiana XylT gene oligonucleotide
gtcaccggagattagaactcactcactat

SEQ ID NO: 13 - DNA, 30 bp, Artificial sequence
  Nicotiana benthamiana XylT gene oligonucleotide
atcagggatccactgcacggtatgctcctc

SEQ ID NO: 14 - DNA, 30 bp, Artificial sequence
  Nicotiana benthamiana XylT gene oligonucleotide
atcgtggtacctagctgcgtctgcaaaaag

SEQ ID NO: 15 - DNA, 35 bp, Artificial sequence
  Nicotiana benthamiana XylT gene oligonucleotide
tatatgaattctagattagcaatgaagagcaagta

SEQ ID NO: 16 - DNA, 27 bp, Artificial sequence
  Nicotiana benthamiana XylT gene oligonucleotide
attgcggtaccgcataagacccctcca

SEQ ID NO: 17 - DNA, 12435 bp, Artificial sequence
  XylT-RNAi construct
ctagattagcaatgaagagcaagtatttgattccataaagagctgggccttaaaccactcggagtgcaaattaaatgtaattagtggattgtttgcccacatgtccatgaaagagcaagttcgagcaatccaagatgcttttgtcattgttggtgctcatggagcaggtctaacccacatagtttctgcagcaccaaaagctgtaatactagaaattataagcagcgaatataggcgcccccattttgctctgattgctcaatggaaaggattggagtaccatcccatatatttggaggggtcttatgcggatccactgcacggtatgctcctcttcttgttcatggtcatgatccttatatgagcagggaaagtccagtttagacttgtagttagttactcttcgttataggatttggatttcttgcgtgtttatggttttagtttccctcctttgatgaataaaattgaatcttgtatgagtttcatatccatgttgtgaatctttttgcagacgcagctaggtaccgcataagacccctccaaatatatgggatggtactccaatcctttccattgagcaatcagagcaaaatgggggcgcctatattcgctgcttataatttctagtattacagcttttggtgctgcagaaactatgtgggttagacctgctccatgagcaccaacaatgacaaaagcatcttggattgctcgaacttgctctttcatggacatgtgggcaaacaatccactaattacatttaatttgcactccgagtggtttaaggcccagctctttatggaatcaaatacttgctcttcattgctaatctagagctcgttaacggtaccatcgatagatctgcgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatatcattctttacaagtatacgtgtttaaattgaataccataaatttttatttttcaaatacatgtaaaattatgaaatgggagtggtggcgaccgagctcaagcacacttcaattcctataacggaccaaatcgcaaaaattataataacatattatttcatcctggattaaaagaaagtcaccggggattattttgtgacgccgattacatacggcgacaataaagacattggaaatcgtagtacatattggaatacactgattatattaatgatgaatacatactttaatatccttacgtaggatcaacatatcttgttacaatcggacacttttgcttcatccccgctaacacctctgcaccttagaccaagcgcttccacaaggaactgagagccatagcccacctcaccttgggttcctttggccgcctgtctttctgaaagagagccttgcccaccgcaactatttcaacacagataggatcaacccgggatggcgctaagaagctattgccgccgatcttcatagggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccggggaccgccgtgttgctaggatggttgttcttggatcagacgctgagtgcgcttcaaatcatcggcgtcctgctcgtgatcgggagtatctggctgggccaacgttccaaccgcactcctagggcgcgtatagcttgccggaagtcgccttgacccgcatggcataggcctatcgtttccacgatcagcgatcggctcgttgccctgcgccgctccaaagcccgcgacgcagcgccggcaggcagagcaagtagagggcagcgcctgcaatccatgcccacccgttccacgttgttatagaagccgcatagatcgccgtgaagaggaggggtccgacgatcgaggtcaggctggtgagcgccgccagtgagccttgcagctgcccctgacgttcctcatccacctgcctggacaacattgcttgcagcgccggcattccgatgccacccgaagcaagcaggaccatgatcgggaacgccatccatccccgtgtcgcgaaggcaagcaggatgtagcctgtgccgtcggcaatcattccgagcatgagtgcccgcctttcgccgagccgggcggctacagggccggtgatcattgcctgggcgagtgaatgcagaatgccaaatgcggcaagcgaaatgccgatcgtggtcgcgtcccagtgaaagcgatcctcgccgaaaatgacccaaagcgcggccggcacctgtccgacaagttgcatgatgaagaagaccgccatcagggcggcgacgacggtcatgccccgggcccaccggaacgaagcgagcgggttgagagcctcccggcgtaacggccggcgttcgcctttgtgcgactccggcaaaaggaaacagcccgtcaggaaattgaggccgttcaaggctgccgcggcgaagaacggagcgtggggggagaaaccgcccatcagcccaccgagcacaggtcccgcgaccatcccgaacccgaaacaggcgctcatgaagccgaagtgccgcgcgcgctcatcgccatcagtgatatcggcaatataagcgccggctaccgccccagtcgccccggtgatgccggccacgatccgcccgatatagagaacccaaaggaaaggcgctgtcgccatgatggcgtagtcgacagtggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcatgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcctgctgcggcgtccgctcgtgggccgtggcgcgggtccgcgcgccggcctcgtgcgcctggcgctcgcgggcgaggtccagggcggccgtcttcacgttctgccttgcgcagatgagatagatccagtctgcaagtgtcagaaacgagtctgcatttgatgccctgaacgtcttcatacatctgataagcccctcccggaagtctgctttctctccgtcagaaagcataccctgaccggtttcaatcacggaccgttctgtcaggtaatgctctgtccagtcatacagagccgggtttgtcacccgtataagctgaagcaggcacaaatcagggaaataaacaaaatcccgcatccccggataaagaaaaatcagggaattaatggcctgatggatttcccgtggcgtcgaaagtgcggcaccataggtatcagtcaccgcgatgagatcccttaccattccagagtctggcggttgattattaatttgctgatatagagcctcagcccgctggcgaaattcattacgtaaatcaaaggcttcaggtcggggtaatttaaaactaagctgaatgattttctggagataacggctgccatcttcgatattcagcgcatgttcaacggcatgagtgataatctgcctgtcataacagagaatatgggtaaagcggggcagatcggctactgcacgcacaagcctgaacacttccgccacctgggatggctccagtcggtccagatcatccatgacaacaatgaacttcagatccagactcaccagttgtcctgcaatttcagcccgaagctagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgtaccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaaaatcgaaatacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggtactgtgtaatgacgatgagcaatcgagaggctgactaacaaaaggtatgcccaaaaacaacctctccaaactgtttcgaattggaagtttctgctcatgccgacaggcataacttagatattcgcgggctattcccactaattcgtcctgctggtttgcgccaagataaatcagtgcatctccttacaagttcctctgtcttgtgaaatgaactgctgactgccccccaagaaagcctcctcatctcccagttggcggcggctgatacaccatcgaaaacccacgtccgaacacttgatacatgtgcctgagaaataggaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaccctcgaccaagctt

SEQ ID NO: 18 - DNA, 21 bp, Artificial sequence
  Nicotiana benthamiana XylT gene oligonucleotide
tgggckgartaygatatyatg

SEQ ID NO: 19 - DNA, 20 bp, Artificial sequence
  Nicotiana benthamiana XylT gene oligonucleotide
gartgyacagcwgccatatc

SEQ ID NO: 20 - DNA, 45 bp, Artificial sequence
  Nicotiana benthamiana XylT gene oligonucleotide
ctaatacgactcactatagggcaagcagtggtatcaacgcagagt

SEQ ID NO: 21 - DNA, 22 bp, Artificial sequence
  Nicotiana benthamiana XylT gene oligonucleotide
ctaatacgactcactatagggc

SEQ ID NO: 22 - DNA, 29 bp, Artificial sequence
  Nicotiana benthamiana XylT gene oligonucleotide
ggattgacccagctaccagagactgaaag

SEQ ID NO: 23 - DNA, 25 bp, Artificial sequence
  Nicotiana benthamiana XylT gene oligonucleotide
ccttggcagcggctttcatttctaa

SEQ ID NO: 24 - DNA, 23 bp, Artificial sequence
  Nicotiana benthamiana XylT gene oligonucleotide
aagcagtggtatcaacgcagagt

SEQ ID NO: 25 - DNA, 29 bp, Artificial sequence
  Nicotiana benthamiana XylT gene oligonucleotide
tatactgcagtggtgctcgcaacttccgt

SEQ ID NO: 26 - DNA, 1503 bp, Nicotiana benthamiana
atgagatcggcgtcaaattcaaacgcacccaataagcaatggcgcaattggttgcctctgttcgttgccctagtgattatagctgagttttcttttctggttcgactcgacgtagctgaaaaagccaactcttgggccgaatcgttttatcagttcaccacggcctcttggtccacctctaaactggctgttgaccacggcgacgttgaggaggtccagttgggtgttttgagtggtgagttcgatcatggcttcgtacctgggagttgcgaggagtggttggaaagggaagattctgtggcttattcgagggattttgataatgaaccaatttttgttcatgggcctggacaggaattgaaatcttgttccataggatgtaagtttggaacagattccaataagaagcctgatgcagcatttcggctaccacaacaagctggcacagctagtgtgctacggtcgatggagtcagctcaatactatgcagagaacaacattactttggcacgacgaaggggatatgatgttgtaatgacaacaagcctctcttcagatgttcctgttggatacttctcttgggctgagtatgatatcatggctccagtagaacctaaaacagagaatgccttggcagcggctttcatttctaattgtggtgctcgcaacttccgtttgcaagctttagaagcccttgaaagggcaaatatcagaattgactcttatggaagttgtcatcataacagggatggaagagttgacaaagtggcagcactgaagcgttaccagtttagcttggcttttgagaattctaatgaggaggactatgtaactgaaaaattctttcagtctctggtagctgggtcaatccctgtggtggttggtgctccaaacatccaagactttgcgccttctcctaattcagttttacacattaaagagataaaagatgctgaatcaattgccaataccatgaagtaccttgctcaaaaccctattgcatataatgagtcattaaggtggaagtttgagggcccatctgatgccttcaaagcccttgttgatatggcagctgttcactcatcttgtcgtttgtgcatcttcttggcaagtaggatccgggaaagagaagagcagagtccaaaatttatgaagcgtccctgcaaatgtaccagagggactgaaactgtatatcatgtatatgtaggtgaaagaggcaggtttgagatggattccattttcttaaggtcgagtgatttgtctttgaaggcgtttgaatctgctatcctctcgaggttcaagtctgttaaacatgttcctgtttggaaggaggaaagacctcaagtactacgaggtggtgatgaactcaaactttacaaagtatatcctgttggcttgacacagagacaagcattgttttccttcagattcaacggggatactgagtttaacaattacattcaaagccacccatgtgcaaaatttgaagccatcttcgtatag

SEQ ID NO: 27 - protein, 500 aa, Nicotiana benthamiana
MRSASNSNAPNKQWRNWLPLFVALVIIAEFSFLVRLDVAEKANSWAESFYQFTTASWSTSKLAVDHGDVEEVQLGVLSGEFDHGFVPGSCEEWLEREDSVAYSRDFDNEPIFVHGPGQELKSCSIGCKFGTDSNKKPDAAFRLPQQAGTASVLRSMESAQYYAENNITLARRRGYDVVMTTSLSSDVPVGYFSWAEYDIMAPVEPKTENALAAAFISNCGARNFRLQALEALERANIRIDSYGSCHHNRDGRVDKVAALKRYQFSLAFENSNEEDYVTEKFFQSLVAGSIPVVVGAPNIQDFAPSPNSVLHIKEIKDAESIANTMKYLAQNPIAYNESLRWKFEGPSDAFKALVDMAAVHSSCRLCIFLASRIREREEQSPKFMKRPCKCTRGTETVYHVYVGERGRFEMDSIFLRSSDLSLKAFESAILSRFKSVKHVPVWKEERPQVLRGGDELKLYKVYPVGLTQRQALFSFRFNGDTEFNNYIQSHPCAKFEAIFV

SEQ ID NO: 28 - DNA, 35 bp, Artificial sequence
  Nicotiana benthamiana XylT gene oligonucleotide
ttatggtaccggatccttggcagcggctttcattt

SEQ ID NO: 29 - DNA, 36 bp, Artificial
  Nicotiana benthamiana XylT gene oligonucleotide
aattggtaccggatccatcagatgggccctcaaact

SEQ ID NO: 30 - DNA, 35 bp, Artificial sequence
  Nicotiana benthamiana XylT gene oligonucleotide
ttatggtacctctagattggcagcggctttcattt

SEQ ID NO: 31 - DNA, 12667 bp, Artificial sequence
  FucT-RNAi construct
ctagagctcgttaacggtaccatcgatagatctgcgatgagctaagctagctatatcatcaatttatgtattacacataatatcgcactcagtctttcatctacggcaatgtaccagctgatataatcagttattgaaatatttctgaatttaaacttgcatcaataaatttatgtttttgcttggactataatacctgacttgttattttatcaataaatatttaaactatatttctttcaagatatcattctttacaagtatacgtgtttaaattgaataccataaatttttatttttcaaatacatgtaaaattatgaaatgggagtggtggcgaccgagctcaagcacacttcaattcctataacggaccaaatcgcaaaaattataataacatattatttcatcctggattaaaagaaagtcaccggggattattttgtgacgccgattacatacggcgacaataaagacattggaaatcgtagtacatattggaatacactgattatattaatgatgaatacatactttaatatccttacgtaggatcaacatatcttgttacaatcggacacttttgcttcatccccgctaacacctctgcaccttagaccaagcgcttccacaaggaactgagagccatagcccacctcaccttgggttcctttggccgcctgtctttctgaaagagagccttgcccaccgcaactatttcaacacagataggatcaacccgggatggcgctaagaagctattgccgccgatcttcatagggtaccgagctcgaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaaatatcatctccctcgcagagatccgaattatcagccttcttattcatttctcgcttaaccgtgacaggctgtcgatcttgagaactatgccgacataataggaaatcgctggataaagccgctgaggaagctgagtggcgctatttctttagaagtgaacgttgacgatcgtcgacggatcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccggggaccgccgtgttgctaggatggttgttcttggatcagacgctgagtgcgcttcaaatcatcggcgtcctgctcgtgatcgggagtatctggctgggccaacgttccaaccgcactcctagggcgcgtatagcttgccggaagtcgccttgacccgcatggcataggcctatcgtttccacgatcagcgatcggctcgttgccctgcgccgctccaaagcccgcgacgcagcgccggcaggcagagcaagtagagggcagcgcctgcaatccatgcccacccgttccacgttgttatagaagccgcatagatcgccgtgaagaggaggggtccgacgatcgaggtcaggctggtgagcgccgccagtgagccttgcagctgcccctgacgttcctcatccacctgcctggacaacattgcttgcagcgccggcattccgatgccacccgaagcaagcaggaccatgatcgggaacgccatccatccccgtgtcgcgaaggcaagcaggatgtagcctgtgccgtcggcaatcattccgagcatgagtgcccgcctttcgccgagccgggcggctacagggccggtgatcattgcctgggcgagtgaatgcagaatgccaaatgcggcaagcgaaatgccgatcgtggtcgcgtcccagtgaaagcgatcctcgccgaaaatgacccaaagcgcggccggcacctgtccgacaagttgcatgatgaagaagaccgccatcagggcggcgacgacggtcatgccccgggcccaccggaacgaagcgagcgggttgagagcctcccggcgtaacggccggcgttcgcctttgtgcgactccggcaaaaggaaacagcccgtcaggaaattgaggccgttcaaggctgccgcggcgaagaacggagcgtggggggagaaaccgcccatcagcccaccgagcacaggtcccgcgaccatcccgaacccgaaacaggcgctcatgaagccgaagtgccgcgcgcgctcatcgccatcagtgatatcggcaatataagcgccggctaccgccccagtcgccccggtgatgccggccacgatccgcccgatatagagaacccaaaggaaaggcgctgtcgccatgatggcgtagtcgacagtggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcatgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcctgctgcggcgtccgctcgtgggccgtggcgcgggtccgcgcgccggcctcgtgcgcctggcgctcgcgggcgaggtccagggcggccgtcttcacgttctgccttgcgcagatgagatagatccagtctgcaagtgtcagaaacgagtctgcatttgatgccctgaacgtcttcatacatctgataagcccctcccggaagtctgctttctctccgtcagaaagcataccctgaccggtttcaatcacggaccgttctgtcaggtaatgctctgtccagtcatacagagccgggtttgtcacccgtataagctgaagcaggcacaaatcagggaaataaacaaaatcccgcatccccggataaagaaaaatcagggaattaatggcctgatggatttcccgtggcgtcgaaagtgcggcaccataggtatcagtcaccgcgatgagatcccttaccattccagagtctggcggttgattattaatttgctgatatagagcctcagcccgctggcgaaattcattacgtaaatcaaaggcttcaggtcggggtaatttaaaactaagctgaatgattttctggagataacggctgccatcttcgatattcagcgcatgttcaacggcatgagtgataatctgcctgtcataacagagaatatgggtaaagcggggcagatcggctactgcacgcacaagcctgaacacttccgccacctgggatggctccagtcggtccagatcatccatgacaacaatgaacttcagatccagactcaccagttgtcctgcaatttcagcccgaagctagcttggctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcggaaagggtcgacgatcttgctgcgttcggatattttcgtggagttcccgccacagacccggattgaaggcgagatccagcaactcgcgccagatcatcctgtgacggaactttggcgcgtgatgactggccaggacgtcggccgaaagagcgacaagcagatcacgcttttcgacagcgtcggatttgcgatcgaggatttttcggcgctgcgctacgtccgcgaccgcgttgagggatcaagccacagcagcccactcgaccttctagccgacccagacgagccaagggatctttttggaatgctgctccgtcgtcaggctttccgacgtttgggtggttgaacagaagtcattatcgcacggaatgccaagcactcccgaggggaaccctgtggttggcatgcacatacaaatggacgaacggataaaccttttcacgcccttttaaatatccgattattctaataaacgctcttttctcttaggtttacccgccaatatatcctgtcaaacactgatagtttaaactgaaggcgggaaacgacaatctgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgtaccataaattcccctcggtatccaattagagtctcatattcactctcaatccaaataatctgcaatggcaattaccttatccgcaacttctttacctatttccgcccggatccgggcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccgatccaacacttacgtttgcaacgtccaagagcaaatagaccacgaacgccggaaggttgccgcagcgtgtggattgcgtctcaattctctcttgcaggaatgcaatgatgaatatgatactgactatgaaactttgagggaatactgcctagcaccgtcacctcataacgtgcatcatgcatgccctgacaacatggaacatcgctatttttctgaagaattatgctcgttggaggatgtcgcggcaattgcagctattgccaaaatcgaaatacccctcacgcatgcattcatcaatattattcatgcggggaaaggcaagattaatccaactggcaaatcatccagcgtgattggtaacttcagttccagcgacttgattcgttttggtgctacccacgttttcaataaggacgagatggtggagtaaagaaggagtgcgtcgaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgatcaaacttcggtactgtgtaatgacgatgagcaatcgagaggctgactaacaaaaggtatgcccaaaaacaacctctccaaactgtttcgaattggaagtttctgctcatgccgacaggcataacttagatattcgcgggctattcccactaattcgtcctgctggtttgcgccaagataaatcagtgcatctccttacaagttcctctgtcttgtgaaatgaactgctgactgccccccaagaaagcctcctcatctcccagttggcggcggctgatacaccatcgaaaacccacgtccgaacacttgatacatgtgcctgagaaataggaacatggtggagcacgacactctcgtctactccaagaatatcaaagatacagtctcagaagaccaaagggctattgagacttttcaacaaagggtaatatcgggaaacctcctcggattccattgcccagctatctgtcacttcatcaaaaggacagtagaaaaggaaggtggcacctacaaatgccatcattgcgataaaggaaaggctatcgttcaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagaggaccctcgaccaagcttctagaggatccttggcagcggctttcatttctaattgtggtgctcgcaacttccgtttgcaagctttagaagcccttgaaagggcaaatatcagaattgactcttatggaagttgtcatcataacagggatggaagagttgacaaagtggcagcactgaagcgttaccagtttagcctggcttttgggaattctaatgaggaggactatgtaactgaaaaattctttcagtctctggtagctgggtcaatccctgtggtggttggtgctccaaacatccaagactttgcgccttctcctaattcagttttacacattaaagagataaaagatgctgaatcaattgccaataccatgaagtaccttgctcaaaaccctattgcatataatgagtcattaaggtggaagtttgagggcccatctgatggatccactgcacggtatgctcctcttcttgttcatggtcatgatccttatatgagcagggaaagtccagtttagacttgtagttagttactcttcgttataggatttggatttcttgcgtgtttatggttttagtttccctcctttgatgaataaaattgaatcttgtatgagtttcatatccatgttgtgaatctttttgcagacgcagctaggtaccggatccatcagatgggccctcaaacttccaccttaatgactcattatatgcaatagggttttgagcaaggtacttcatggtattggcaattgattcagcatcttttatctctttaatgtgtaaaactgaattaggagaaggcgcaaagtcttggatgtttggagcaccaaccaccacagggattgacccagctaccagagactgaaagaatttttcagttacatagtcctcctcattagaattcccaaaagccaggctaaactggtaacgcttcagtgctgccactttgtcaactcttccatccctgttatgatgacaacttccataagagtcaattctgatatttgccctttcaagggcttctaaagcttgcaaacggaagttgcgagcaccacaattagaaatgaaagccgctgccaat

(19) United States
(12) Patent Application Publication
(10) Pub. No.: US 2010/0242128 A1
(43) Pub. Date: Sep. 23, 2010

(54) METHOD TO PRODUCE MODIFIED PLANTS WITH ALTERED N-GLYCOSYLATION PATTERN

(75) Inventors: Herta Steinkellner, Vienna (AT); Richard Strasser, Vienna (AT); Josef Glössl, Vienna (AT); Koen Weterings, Raleigh, NC (US); Gerben Van Eldik, Zwijnaarde (BE)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(73) Assignee: Bayer BioSceince NV, Gent (BE)

(21) Appl. No.: 12/740,433

(22) PCT Filed: Oct. 31, 2007
(86) PCT No.: PCT/EP07/09455

§ 371 Date: Apr. 29, 2010

(51) Int. Cl. A01H 1/02; C12N 5/04; C12N 5/10; A01H 1/00; C12Q 1/02; C07K 14/00; C12Q 1/68; C07H 21/04; A01H 5/00; A01H 5/10

(52) U.S. Cl. 800/260; 435/410; 435/414; 435/419; 800/278; 435/29; 530/395; 435/6; 536/23.6; 800/317.3

(57) ABSTRACT

Provided is a novel method to produce a plant cell or plant having an altered N-glycosylation pattern resulting, in particular, in a low level of β-1,2-xylose residues and core α-1,3-fucose residues on protein-bound N-glycans. The plant cell or plant is of particular interest for producing therapeutic glycoproteins with a low, or not detectable, level of β-1,2-xylose and α-1,3-fucose residues. Also provided are novel α-1,3-fucosyltransferase nucleotide sequences and uses.

The following invention relates to a method to produce modified plants, particularly Nicotiana plants such as Nicotiana benthamiana, which have an altered N-glycosylation pattern resulting in a lower level of immunogenic protein-bound N-glycans, particularly a lower level of β-1,2-xylose residues and core α-1,3-fucose residues on the protein-bound N-glycans, than counterpart unmodified plants. The modified plants may even have no detectable immunogenic protein-bound N-glycans, particularly no detectable β-1,2-xylose residues and core α-1,3-fucose residues on the protein-bound N-glycans. Such plants may be obtained by providing modified plants having a lower expression of the endogenous β-1,2-xylosyltransferase encoding gene(s) and providing modified plants having a lower expression of the endogenous α-1,3-fucosyltransferase encoding gene(s), and further crossing both of said modified plants.

### DESCRIPTION OF RELATED ART

The use of transgenic plants for the production of value-added recombinant proteins, such as antibodies, vaccines, human blood products, hormones, growth regulators and the like, is described to offer many practical, economic and safety advantages compared with more conventional systems such as animal and insect cell cultures, yeast, filamentous fungi and bacteria (reviewed by Stoger et al. (2002) Curr. Opin. Biotechnol. 13: 161-166; Twyman et al. (2003) Trends Biotechnol. 21: 570-578; Fischer et al. (2004) Curr. Opin. Plant Biol. 7: 152-158).

Although the protein synthesis pathway is largely the same in plants and animals, there are some differences in posttranslational modifications, particularly with respect to glycan-chain structures. Thus, plant-derived recombinant human proteins tend to have the carbohydrate groups β-1,2-xylose and α-1,3-fucose, which are absent in mammals, but lack the terminal galactose and sialic acid residues that are found on many native human glycoproteins (Twyman et al. (2003) Trends Biotechnol. 21: 570-578).

The immunogenicity of β-1,2-xylose and core α-1,3-fucose residues in mammals is well documented (Kurosaka et al. (1991) J. Biol. Chem. 266: 4168-4172; Faye et al. (1993) Analytical Biochemistry 209: 104-108; Bardor et al. (2003) Glycobiology 13: 427-434; Bencurova et al. (2004) Glycobiology 14: 457-466; Jin et al. (2006) Glycobiology 16: 349-357). Furthermore, altered N-glycosylation may affect the function of a protein (Jefferis (2005) Biotechnol. Prog. 21: 11-16), thus, the unique N-glycans added by plants could impact on both immunogenicity and functional activity of the protein and, consequently, may represent a limitation for plants to be used as a protein production platform.

The enzyme that catalyses the transfer of xylose from UDP-xylose to the core β-linked mannose of protein-bound N-glycans is β-1,2-xylosyltransferase (“XylT”, EC 2.4.2.38). The β-1,2-xylosyltransferase is an enzyme unique to plants and some non-vertebrate animal species, e.g. in Schistosoma species (Khoo et al. (1997) Glycobiology 7: 663-677) and snail (e.g. Mulder et al. (1995) Eur. J. Biochem. 232: 272-283) and does not occur in human beings or in other vertebrates. Tezuka et al. (Eur. J. Biochem. (1992) 203(3): 401-413) characterized a β-1,2-xylosyltransferase of sycamore (Acer pseudoplatanus L.). Zeng et al. (J. Biol. Chem. (1997) 272: 31340-31347) described the purification of a β-1,2-xylosyltransferase from soybean microsomes. Only a part of the soybean β-1,2-xylosyltransferase cDNA was isolated (WO99/29835). Strasser et al. (FEBS Lett. (2000) 472:105-108) and WO01/64901 described the isolation of an Arabidopsis XylT gene, the predicted amino acid sequence of the encoded XylT protein and its enzymatic activity in vitro and in vivo. WO07/107,296 described the isolation of XylT gene variants from Nicotiana benthamiana and Nicotiana tabacum, and the predicted amino acid sequence of the encoded XylT proteins.

Genes encoding β-1,2-xylosyltransferase in plants are well known and include the following database entries identifying experimentally demonstrated and putative XylT cDNA and gene sequences, parts thereof or homologous sequences: AJ627182, AJ627183 (Nicotiana tabacum cv. Xanthi), AM179855 (Solanum tuberosum), AM179856 (Vitis vinifera), AJ891042 (Populus alba×Populus tremula), AY302251 (Medicago sativa), AJ864704 (Saccharum officinarum), AM179857 (Zea mays), AM179853 (Hordeum vulgare), AM179854 (Sorghum bicolor), BD434535, AJ277603, AJ272121, AF272852, AX236965 (Arabidopsis thaliana), AJ621918 (Oryza sativa), AR359783, AR359782, AR123000, AR123001 (Soybean), AJ618933 (Physcomitrella patens), as well as the nucleotide sequences from Nicotiana species described in application PCT/EP2007/002322 (all sequences herein incorporated by reference).

The enzyme that catalyses the transfer of fucose from GDP-fucose to the core β-linked N-acetyl glucosamine (GlcNAc) of protein-bound N-glycans is α-1,3-fucosyltransferase (“FucT”, EC 2.4.1.214).

Genes encoding α-1,3-fucosyltransferase in plants are well known and include the following database entries identifying experimentally demonstrated and putative FucT cDNA and gene sequences, parts thereof or homologous sequences: NM112815, NM103858, At1g49710, At3g19280, AJ345084, AJ345085, AF154111, NM106102 (Arabidopsis thaliana), AJ618932, AJ429145 (Physcomitrella patens), DQ789145 (Lemna minor), AY557602 (Medicago truncatula), Y18529 encoding protein Q9ST51 (Vigna radiata), AP004457, AK099681 encoding protein AAS66306.1 (Oryza sativa), AJ891040 encoding protein CAI70373 (Populus alba×Populus tremula) AY082445 encoding protein AAL99371, AY082444 encoding protein AAL99370 (Medicago sativa), AJ582182 encoding protein CAE46649 (Triticum aestivum) AJ582181 encoding protein CAE46648 (Hordeum vulgare), AY964641 (Zea mays) (all sequences herein incorporated by reference).

Various strategies have been applied to avoid plant specific N-glycosylation of the proteins produced by plants.

One strategy, based on targeting of proteins to specific subcellular compartments with defined N-glycan structures, was reported (Schouten et al. (1996) Plant Mol. Biol. 30: 781-793). For example, retention of recombinant proteins in the endoplasmic reticulum resulted in the accumulation of proteins carrying mainly oligo-mannosidic N-glycans, which are typical for endoplasmic reticulum resident proteins. However, these structures may lead to a dramatic reduction of the in vivo half life of the target protein as reported for a plant produced antibody (Ko et al. (2003) PNAS 100: 8013-8018).

Another promising strategy is based on the specific manipulation of the N-glycosylation pathway in host plants. The overexpression of human β-1,4-galactosyltransferase, which competes for the same acceptor substrate as β-1,2-xylosyltransferase and core α-1,3-fucosyltransferase, resulted in a significant reduction of β-1,2-xylose and core α-1,3-fucose (Palacpac et al. (1999) PNAS 96: 4692-4697; Bakker et al. (2001) PNAS 98: 2899-2904; Bakker et al. (2006) PNAS 103: 7577-7582). However, the complete elimination of these glycan epitopes has not been achieved. On the other hand it was possible to partially elongate plant N-glycans with β-1,4-galactose, a terminal residue present on many mammalian N-linked glycans but absent in plants.

Recently, a knockout line from the model plant A. thaliana was generated, with deficiency of active β-1,2-xylosyltransferase (XylT) and core α-1,3-fucosyltransferase (FucT), the enzymes responsible for the transfer of β-1,2-xylose and core α-1,3-fucose (Strasser et al. (2004) FEBS Lett. 561: 132-136). Endogenous glycoproteins from this line (“XylT/FucT knock-out line”) lack immunogenic β-1,2-xylose and core α-1,3-fucose residues. These XylT/FucT knock-out plants are viable and revealed no obvious morphological phenotype under standard growth conditions. Similar results were obtained with the moss Physcomitrella patens after disruption of the XylT and FucT genes by homologous recombination (Koprivova et al. (2004) Plant Biotech. J. 2: 517-523).

Alternatively, a RNA interference (RNAi) strategy has been applied to eliminate xylose and fucose residues in the aquatic plant Lemna minor (Cox et al. (2006) Nature Biotechnol. 24: 1591-1597). Optimization of glycosylation was accomplished by co-expression with a single RNAi transcript designed to silence endogenous L. minor β-1,2-xylosyltransferase and α-1,3-fucosyltransferase activities. An IgG produced in these RNAi plants exhibited a homogenous complex N-glycan (GnGn) structure without xylose and fucose residues.

Leafy crops, such as tobacco, are considered to be strong candidates for the commercial production of recombinant proteins (see e.g. Twyman et al. (2003) Trends Biotechnol. 21: 570-578).

The aim of the current invention is to provide alternative methods for producing modified plants, particularly Nicotiana plants including Nicotiana benthamiana plants, which have a lower level or altered pattern of protein-bound N-glycans, particularly a lower level of β-1,2-xylose residues and core α-1,3-fucose residues on protein-bound N-glycans, than counterpart unmodified plants, as well as DNA fragments to carry out such methods. More particularly, the modified plants of the invention have no β-1,2-xylose residues and core α-1,3-fucose residues on protein-bound N-glycans.

### SUMMARY OF THE INVENTION

The first object of the invention is a method to produce a plant cell or plant having a low level of β-1,2-xylose residues and core α-1,3-fucose residues on protein-bound N-glycans comprising the steps of:
1) Providing a first plant having a low level of β-1,2-xylose residues on protein-bound N-glycans;
2) Providing a second plant having a low level of core α-1,3-fucose residues on protein-bound N-glycans;
3) Crossing the first plant of step 1) with the second plant of step 2);
4) Optionally, identifying from the progeny obtained from the crossing of step 3) a plant which has a low level of β-1,2-xylose and core α-1,3-fucose residues on protein-bound N-glycans;
   wherein at least one gene encoding a β-1,2-xylosyltransferase in said first plant and at least one gene encoding an α-1,3-fucosyltransferase in said second plant have not been disrupted, deleted, or inactivated by mutagenesis such as substitution, deletion or insertion.

According to a particular aspect of the invention, the plant of step 3) exhibits no detectable β-1,2-xylose residues and no detectable core α-1,3-fucose residues on foreign glycoproteins, such as an antibody.

In one embodiment of the method of the invention, the low level of β-1,2-xylose residues on protein-bound N-glycans in the first plant is achieved by transcriptional or post-transcriptional silencing of the expression of the endogenous β-1,2-xylosyltransferase encoding gene (“XylT” gene); and the low level of α-1,3-fucose residues on protein-bound N-glycans in the second plant is achieved by transcriptional or post-transcriptional silencing of the expression of the endogenous α-1,3-fucosyltransferase encoding gene (“FucT” gene).

In another embodiment of the method of the invention, silencing of XylT gene expression in said first plant is carried out by transforming a plant cell with a first chimeric gene to generate transgenic plant cells, said first chimeric gene comprising the following operably linked DNA fragments: i) a plant expressible promoter, ii) a DNA region which, when transcribed, yields an RNA molecule capable of forming a double stranded RNA region at least between (1) an RNA region transcribed from a first sense DNA region comprising a nucleotide sequence of at least 18 out of 20-21 consecutive nucleotides selected from a nucleotide sequence encoding a XylT protein, or the complement thereof, said nucleotide sequence preferably obtainable from the same species or cultivar as the plant cells into which the first chimeric gene is to be introduced, or selected from a nucleotide sequence of a XylT gene or a XylT cDNA, or the complement thereof, said nucleotide sequence preferably obtainable from the same species or cultivar as the plant cells into which the first chimeric gene is to be introduced; and (2) an RNA region transcribed from a second antisense DNA region comprising a nucleotide sequence of at least 18 consecutive nucleotides which have at least 95% sequence identity to the complement of said first sense DNA region, and iii) a DNA region comprising a transcription termination and polyadenylation signal functional in plants; and silencing of FucT gene expression in said second plant is carried out by transforming a plant cell with a second chimeric gene to generate transgenic plant cells, said second chimeric gene comprising the following operably linked DNA fragments: i) a plant expressible promoter, ii) a DNA region which, when transcribed, yields an RNA molecule capable of forming a double stranded RNA region at least between (1) an RNA region transcribed from a third sense DNA region comprising a nucleotide sequence of at least 18 out of 20-21 consecutive nucleotides selected from a nucleotide sequence encoding a FucT protein, or the complement thereof, said nucleotide sequence preferably obtainable from the same species or cultivar as the plant cells into which the second chimeric gene is to be introduced, or selected from a nucleotide sequence of a FucT gene or a FucT cDNA, or the complement thereof, said nucleotide sequence preferably obtainable from the same species or cultivar as the plant cells into which the second chimeric gene is to be introduced; and (2) an RNA region transcribed from a fourth antisense DNA region comprising a nucleotide sequence of at least 18 consecutive nucleotides which have at least 95% sequence identity to the complement of said third sense DNA region, and iii) a DNA region comprising a transcription termination and polyadenylation signal functional in plants.

In another embodiment, silencing of XylT gene expression in said first plant is carried out by transforming a plant cell with a first chimeric gene to generate transgenic plant cells, said first chimeric gene comprising the following operably linked DNA fragments: i) a plant expressible promoter, ii) a DNA region comprising a nucleotide sequence of at least 18 out of 20-21 consecutive nucleotides selected from a nucleotide sequence encoding a XylT protein, or the complement thereof, said nucleotide sequence preferably obtainable from the same species or cultivar as the plant cells into which said first chimeric gene is to be introduced, or selected from a nucleotide sequence of a XylT gene or a XylT cDNA, or the complement thereof, said nucleotide sequence preferably obtainable from the same species or cultivar as the plant cells into which said chimeric gene is to be introduced, in antisense or sense orientation; and iii) a DNA region comprising a transcription termination and polyadenylation signal functional in plants; while silencing of FucT gene expression in said second plant is carried out by transforming a plant cell with a second chimeric gene to generate transgenic plant cells, said second chimeric gene comprising the following operably linked DNA fragments: i) a plant expressible promoter; ii) a DNA region comprising a nucleotide sequence of at least 18 out of 20-21 consecutive nucleotides selected from a nucleotide sequence encoding a FucT protein, or the complement thereof, said nucleotide sequence preferably obtainable from the same species or cultivar as the plant cells into which said second chimeric gene is to be introduced, or selected from a nucleotide sequence of a FucT gene or a FucT cDNA, or the complement thereof, said nucleotide sequence preferably obtainable from the same species or cultivar as the plant cells into which said second chimeric gene is to be introduced, in the antisense or sense orientation; and iii) a DNA region comprising a transcription termination and polyadenylation signal functional in plants.

In a particular embodiment of the method of the invention, silencing of XylT gene expression in said first plant is carried out by providing one or more first double stranded RNA molecules to said first plant or cells of said first plant, wherein the first double stranded RNA molecule(s) comprise two RNA strands, one RNA strand consisting essentially of an RNA nucleotide sequence of at least 18 out of 20-21 consecutive nucleotides selected from a nucleotide sequence encoding a XylT protein, or the complement thereof, said nucleotide sequence preferably obtainable from the same species or cultivar as the cells of the plant into which the first double stranded RNA molecule(s) is to be introduced, or selected from the nucleotide sequence of a XylT gene or a XylT cDNA, or the complement thereof, said nucleotide sequence preferably obtainable from the same species or cultivar as the plant cells into which said first double stranded RNA molecule(s) is to be introduced; and silencing of FucT gene expression in said second plant is carried out by providing one or more second double stranded RNA molecules to said second plant or cells of said second plant, wherein the second double stranded RNA molecules comprise two RNA strands, one RNA strand consisting essentially of an RNA nucleotide sequence of at least 18 out of 20-21 consecutive nucleotides selected from a nucleotide sequence encoding a FucT protein, or the complement thereof, said nucleotide sequence preferably obtainable from the same species or cultivar as the cells of the plant into which the second double stranded RNA molecule(s) is to be introduced, or selected from the nucleotide sequence of a FucT gene or a FucT cDNA, or the complement thereof, said nucleotide sequence preferably obtainable from the same species or cultivar as the plant cells into which said second double stranded RNA molecule(s) is to be introduced.

Another object of the invention relates to a method to identify a Nicotiana FucT DNA fragment, comprising the steps of: i) providing genomic DNA or cDNA obtainable from a Nicotiana species or cultivar; ii) selecting a means from the following group: a DNA fragment comprising a nucleotide sequence encoding the amino acid sequence of SEQ ID No.: 27, for use as a probe; a DNA fragment comprising the nucleotide sequence of SEQ ID No.: 26, for use as a probe; a DNA fragment or oligonucleotide comprising a nucleotide sequence consisting of between 20 to 200 consecutive nucleotides selected from a nucleotide sequence encoding the amino acid sequence of SEQ ID No.: 27, for use as a probe; a DNA fragment or oligonucleotide comprising a nucleotide sequence consisting of between 20 to 1503 consecutive nucleotides selected from a nucleotide sequence encoding the amino acid sequence of SEQ ID No.: 27, for use as a probe; a DNA fragment or oligonucleotide comprising a nucleotide sequence consisting of between 20 to 200 consecutive nucleotides selected from a nucleotide sequence of SEQ ID No.: 26, for use as a probe; a DNA fragment or oligonucleotide comprising a nucleotide sequence consisting of between 20 to 1503 consecutive nucleotides selected from a nucleotide sequence of SEQ ID No.: 26, for use as a probe; an oligonucleotide sequence having a nucleotide sequence comprising between 20 to 200 consecutive nucleotides selected from a nucleotide sequence encoding the amino acid sequence of SEQ ID No.: 27, for use as a primer in a PCR reaction; an oligonucleotide sequence having a nucleotide sequence comprising between 20 to 200 consecutive nucleotides selected from the nucleotide sequence of SEQ ID No.: 26, for use as a primer in a PCR reaction; or an oligonucleotide having the nucleotide sequence of any one of SEQ ID NO.: 28 and SEQ ID NO.: 29, for use as a primer in a PCR reaction; and iii) identifying a FucT DNA fragment from said Nicotiana species or cultivar by performing a PCR reaction using said genomic DNA or said cDNA and said primers, or by performing hybridization using said genomic DNA or said cDNA and said probes. The identified fragment may subsequently be isolated and used to obtain a Nicotiana plant cell or plant having a low level of α-1,3-fucose residues on protein-bound N-glycans.

In the above-described method to identify a Nicotiana FucT DNA fragment, the DNA fragment or oligonucleotide selected in step ii) preferentially comprises at least one Nicotiana-specific FucT nucleotide and/or encodes at least one Nicotiana-specific FucT amino acid.

It is yet another object of the invention to provide a method to identify a Nicotiana FucT allele correlated with a low level of α-1,3-fucose residues on protein-bound N-glycans comprising the steps of:
a) providing a population, optionally a mutagenized population, of different plant lines of a Nicotiana species or cultivar;
b) identifying in each plant line of said population a Nicotiana FucT DNA fragment according to one of the methods described above;
c) analyzing the level of α-1,3-fucose residues on protein-bound N-glycans of each plant line of said population and identifying those plant lines having a lower level of α-1,3-fucose residues on protein-bound N-glycans than other plant lines;
d) correlating the low level of α-1,3-fucose residues on protein-bound N-glycans in a plant line to the presence of a specific Nicotiana FucT allele.

The identified Nicotiana FucT allele may be introduced in a Nicotiana plant cell or plant of choice to obtain a Nicotiana plant cell or plant having a low level of α-1,3-fucose residues on protein-bound N-glycans.

It is yet another object of the invention to provide an isolated DNA fragment encoding a FucT protein of amino acid sequence SEQ ID NO.: 27 or an isolated DNA fragment comprising the nucleotide sequence of SEQ ID NO.: 26; or any part thereof comprising at least 20, at least 21, at least 22, at least 25, at least 50, at least 100, at least 150, or at least 200 contiguous nucleotides, wherein said part preferentially comprises at least one Nicotiana-specific FucT nucleotide and/or encodes at least one Nicotiana-specific FucT amino acid.

The invention also provides a chimeric gene comprising the following operably linked DNA fragments: (1) a plant expressible promoter; (2) a DNA region which, when transcribed, yields an RNA molecule capable of forming a double stranded RNA region by base-pairing between at least: (i) an RNA region transcribed from a first DNA region comprising at least 18 out of 20, at least 18 out of 21, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in antisense orientation; and (ii) an RNA region transcribed from a second DNA region comprising at least 18 out of 20, at least 18 out of 21, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in sense orientation; and (3) a DNA region comprising a transcription termination and polyadenylation signal functional in plants.

The invention further provides a chimeric gene comprising the following operably linked DNA fragments: (1) a plant expressible promoter; (2) a DNA region comprising at least 18 out of 20, at least 18 out of 21, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in sense or antisense orientation; and (3) a DNA region comprising a transcription termination and polyadenylation signal functional in plants.

Nicotiana plant cells comprising such chimeric genes and Nicotiana plants consisting essentially of such Nicotiana plant cells, as well as seed thereof are also provided by the invention.

Another object of the invention is a method to produce a foreign glycoprotein of interest having a low level, or no detectable, β-1,2-xylose and α-1,3-fucose residues on protein-bound N-glycans, comprising the main steps of (i) producing a plant cell or plant having a low level of β-1,2-xylose residues and core α-1,3-fucose residues on protein-bound N-glycans by carrying out one of the methods according to the invention; (ii) providing to the obtained plant cell or plant a chimeric gene comprising a DNA region encoding the glycoprotein of interest; (iii) cultivating the plant or plant cell obtained in the previous step and, (iv) optionally, extracting and purifying the foreign glycoprotein of interest from the plant proteins.

The invention also relates to the use of:
1) a nucleotide sequence encoding a XylT protein comprising the amino acid sequence of SEQ ID No.: 10, or any part thereof comprising at least 18, at least 19, at least 20, at least 21, at least 22, at least 25, at least 50, at least 100, at least 150, or at least 200 contiguous nucleotides, or a nucleotide sequence comprising the nucleotide sequence of SEQ ID NO.: 9, or any part thereof comprising at least 18, at least 19, at least 20, at least 21, at least 22, at least 25, at least 50, at least 100, at least 150, or at least 200 contiguous nucleotides, to decrease the level of β-1,2-xylose residues on protein-bound N-glycans in a Nicotiana plant; and
2) A nucleotide sequence encoding a FucT protein comprising the amino acid sequence of SEQ ID NO.: 27, or any part thereof comprising at least 18, at least 19, at least 20, at least 21, at least 22, at least 25, at least 50, at least 100, at least 150, or at least 200 contiguous nucleotides, or a nucleotide sequence comprising the nucleotide sequence of SEQ ID NO.: 26, or any part thereof comprising at least 18, at least 19, at least 20, at least 21, at least 22, at least 25, at least 50, at least 100, at least 150, or at least 200 contiguous nucleotides, to decrease the level of α-1,3-fucose residues on protein-bound N-glycans in a Nicotiana plant;
   to obtain, after crossing the Nicotiana plant obtained under 1) with the Nicotiana plant obtained under 2), a Nicotiana plant cell or plant having a low level of β-1,2-xylose residues and α-1,3-fucose residues on protein-bound N-glycans and/or no detectable β-1,2-xylose residues and no detectable α-1,3-fucose residues on N-glycans bound to a foreign glycoprotein.

The invention also relates to the use of a nucleotide sequence encoding a FucT protein comprising the amino acid sequence of SEQ ID NO.: 27, or any part thereof comprising at least 20, at least 21, at least 22, at least 25, at least 50, at least 100, at least 150, or at least 200 contiguous nucleotides, or use of a nucleotide sequence comprising the nucleotide sequence of SEQ ID NO.: 26, or any part thereof comprising at least 20, at least 21, at least 22, at least 25, at least 50, at least 100, at least 150, or at least 200 contiguous nucleotides, to identify a FucT gene or FucT cDNA in a Nicotiana species or cultivar, or to identify an allele of a FucT gene correlated with a low level of α-1,3-fucose residues on protein-bound N-glycans in a Nicotiana species or cultivar, or to introduce an allele of a FucT gene correlated with a low level of α-1,3-fucose residues on protein-bound N-glycans in a Nicotiana species or cultivar. Preferentially, said part of nucleotide sequence and/or said part of amino acid sequence comprises at least one Nicotiana-specific FucT nucleotide and/or encodes at least one Nicotiana-specific FucT amino acid, respectively.

The methods and means described herein are believed to be suitable for all plant cells and plants. However, preferred plants belong to any Nicotiana species or cultivar, in particular Nicotiana benthamiana.

With the foregoing and other objects, advantages and features of the invention that will become hereinafter apparent, the nature of the invention may be more clearly understood by reference to the following detailed description of different embodiments of the invention, the appended claims and the figures.

### BRIEF DESCRIPTION OF THE FIGURES

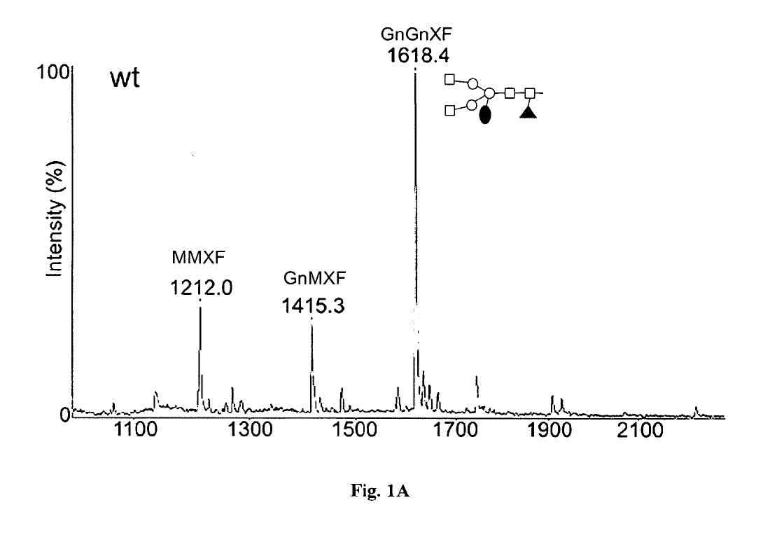

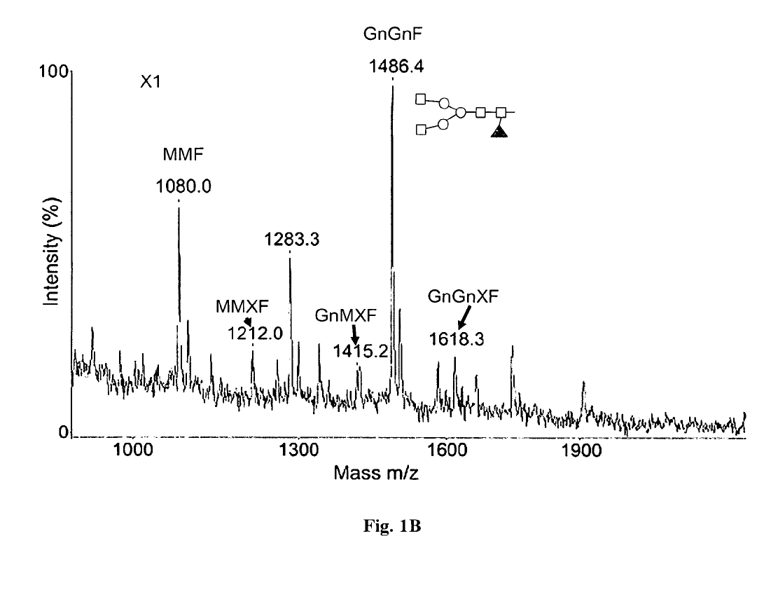

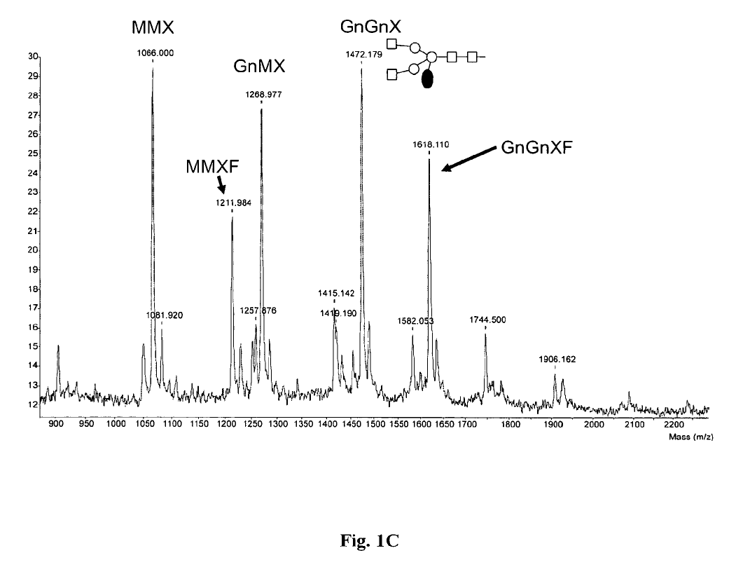

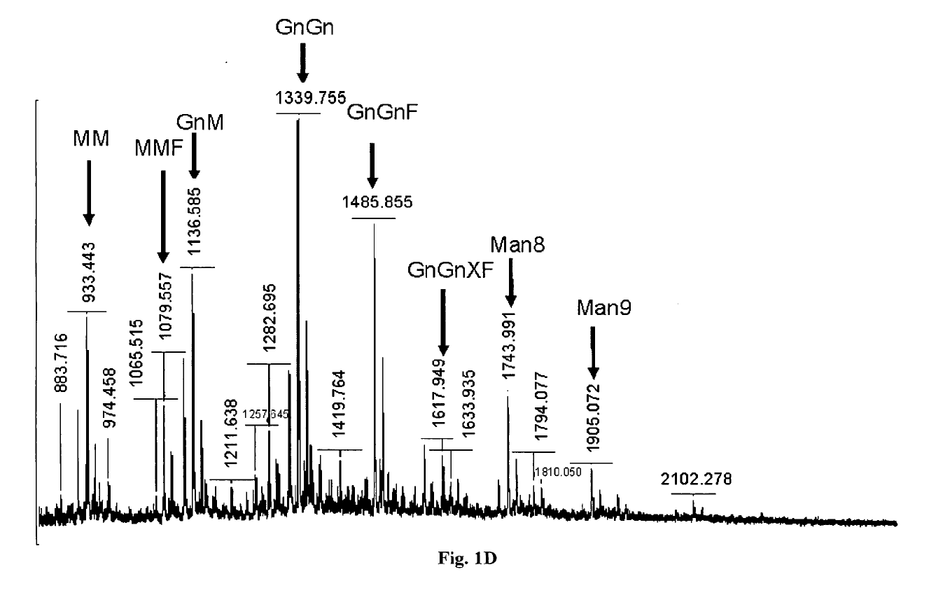

FIG. 1: MALDI-TOF mass spectrometric analysis of N-glycans bound to the soluble endogenous proteins of:
a wild-type line of Nicotiana benthamiana (control) (FIG. 1A)
a XylT-RNAi line of Nicotiana benthamiana (X1) (FIG. 1B)
a FucT-RNAi line of Nicotiana benthamiana (F3) (FIG. 1C)
a XylT-FucT-RNAi line of Nicotiana benthamiana (C100) (FIG. 1D)

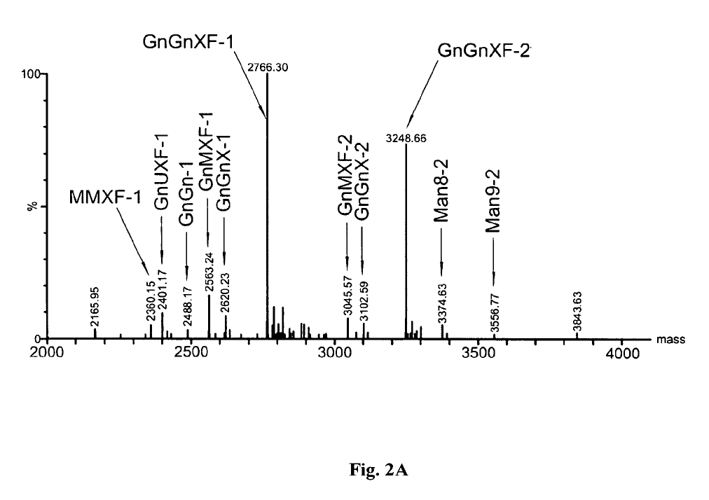

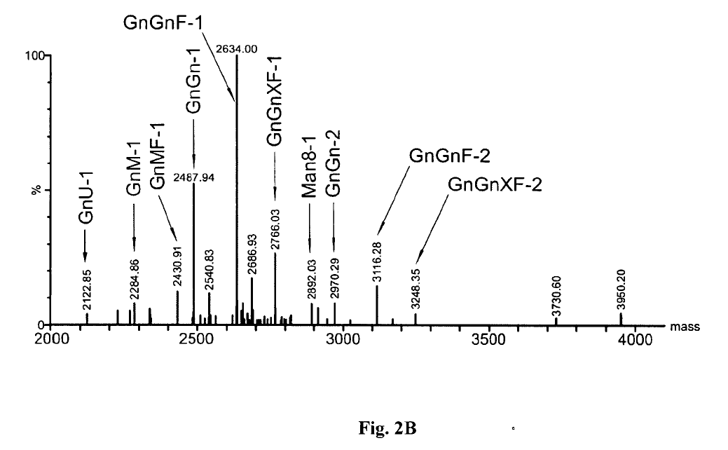

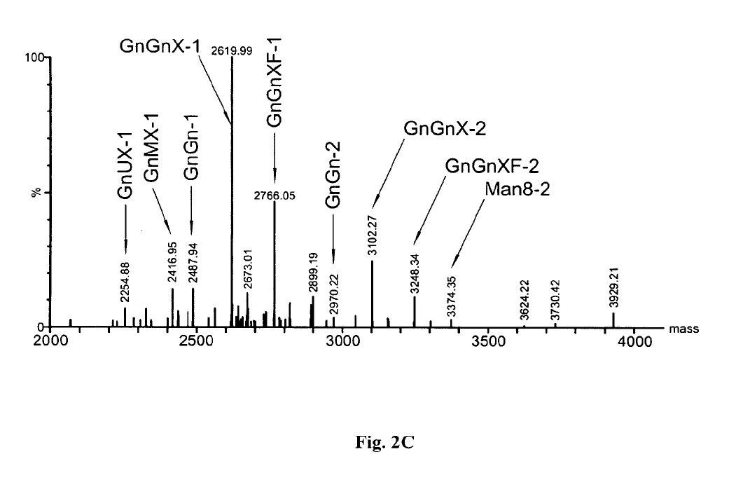

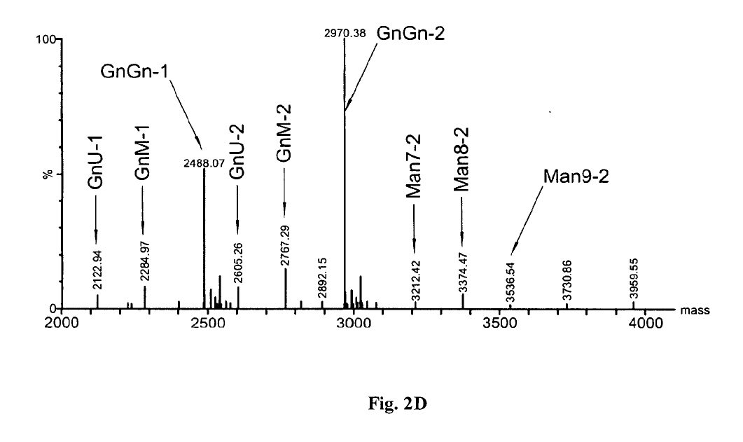

FIG. 2: LC/ESI/MS mass spectrometric analysis of N-glycans bound to the heavy chain of an IgG antibody transiently expressed in:
a wild-type line of Nicotiana benthamiana (control) (FIG. 2A)
a XylT-RNAi line of Nicotiana benthamiana (X1) (FIG. 2B)
a FucT-RNAi line of Nicotiana benthamiana (F3) (FIG. 2C)
a XylT-FucT-RNAi line of Nicotiana benthamiana (C100) (FIG. 2D)

In the figures and along the description, reference is made to the N-glycans abbreviations which are explained in Table 1.

TABLE 1
Structure of N-glycans (See also http://www.proglycan.com for a current
nomenclature of N-glycans). * indicates the bond between the indicated sugar
chain and an asparagine of the peptidic part of the resulting glycoprotein.

| | |
|---|---|
| GnGn | 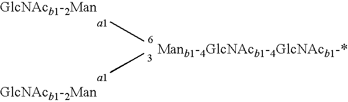 |
| GnGnF | 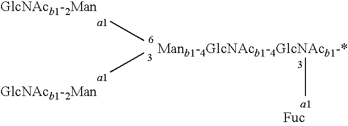 |
| GnGnX | 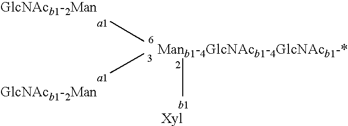 |
| GnGnXF | 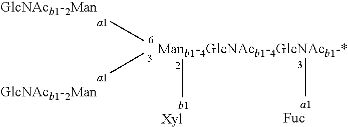 |
| GnM | 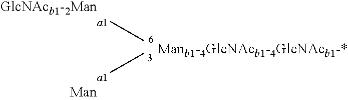 |
| GnMF | 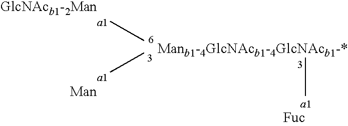 |
| GnMX | 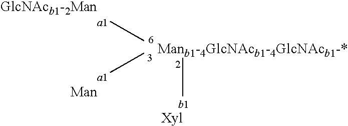 |
| GnMXF | 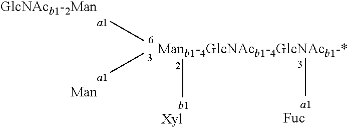 |
| GnU | 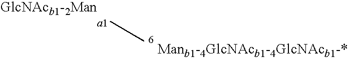 |
| GnUX | 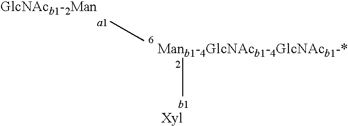 |
| GnUXF | 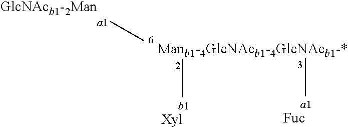 |
| Man7 | 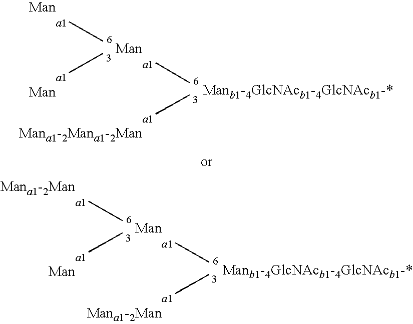 |
| Man8 | 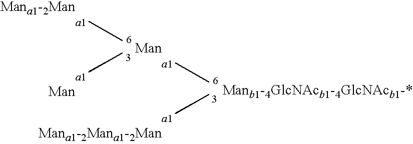 |
| Man9 | 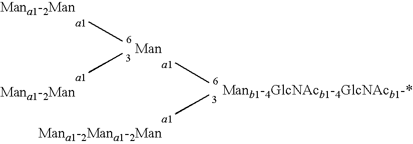 |
| MM | 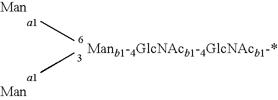 |
| MMF | 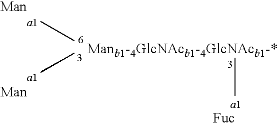 |
| MMX | 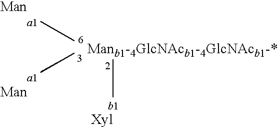 |
| MMXF | 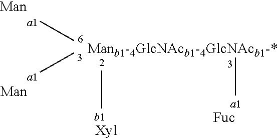 |

### DETAILED DESCRIPTION OF DIFFERENT EMBODIMENTS OF THE INVENTION

The current invention is based on the finding that crossing a first parental plant having a low level of β-1,2-xylose residues on protein-bound N-glycans with a second parental plant having a low level of core α-1,3-fucose residues on protein-bound N-glycans can result in a plant producing glycoproteins having an altered N-glycans profile. Surprisingly, it was found that the N-glycans bound to a glycoprotein produced in a plant obtained after such a crossing have a lower level of β-1,2-xylose residues and core α-1,3-fucose residues in comparison to the levels observed in each of the two parental plants. Even more unexpected was that this effect on the N-glycosylation pattern of a foreign glycoprotein is greater than the sum of the reduction in the level of β-1,2-xylose residues bound to said foreign glycoprotein provided by the first parental plant and the reduction in the level of core α-1,3-fucose residues bound to said foreign glycoprotein provided by the second parental plant. Still more surprisingly, a plant resulting from such a crossing can even produce foreign glycoproteins, such as antibodies, having no detectable β-1,2-xylose and α-1,3-fucose residues on protein-bound N-glycans, while the parental plants produced foreign glycoproteins, such as antibodies, carrying β-1,2-xylose residues and core α-1,3-fucose residues, respectively.

In one embodiment, the invention is related to a method to produce a plant cell or plant having a low level of β-1,2-xylose residues and core α-1,3-fucose residues on protein-bound N-glycans comprising the steps of:
1) Providing a first plant having a low level of β-1,2-xylose residues on protein-bound N-glycans;
2) Providing a second plant having a low level of core α-1,3-fucose residues on protein-bound N-glycans;
3) Crossing the first plant of step 1) with the second plant of step 2);
4) Optionally, identifying from the progeny obtained from the crossing of step 3) a plant which has a low level of β-1,2-xylose and core α-1,3-fucose residues on protein-bound N-glycans;
   wherein at least one gene encoding a β-1,2-xylosyltransferase in said first plant and at least one gene encoding an α-1,3-fucosyltransferase in said second plant have not been disrupted, deleted, or inactivated by mutagenesis such as substitution, deletion or insertion.

As used herein “a plant having a low level of β-1,2-xylose residues” on protein-bound N-glycans is a plant (particularly a Nicotiana plant or a Nicotiana benthamiana plant), in which the β-1,2-xylosyltransferase activity is decreased in comparison to a control plant, resulting in a lower level of β-1,2-xylose residues in comparison to the level of β-1,2-xylose residues on protein-bound N-glycans of the control plant. The “control” plant is generally a selected target plant which could be used as a biofactory for producing therapeutic glycoproteins. Although such a control plant may be any plant, it may advantageously be selected among tobacco and related species like Nicotiana, including N. benthamiana, N. tabacum, and S. tuberosum, or other plants such as M. sativa. Generally, the control plant is an unmodified plant that has not been provided either with a silencing nucleic acid molecule targeted to the endogenous β-1,2-xylosyltransferase encoding gene (“XylT” gene) or with a XylT allele associated with a low level of β-1,2-xylosyltransferase activity. A “plant having a low level of β-1,2-xylose residues” on protein-bound N-glycans is a plant in which the fraction of protein-bound N-glycans having β-1,2-xylose residues represents less than about 50%, especially less than about 30%, especially less than about 20%, especially less than about 15%, more especially less than about 10%; still more especially less than about 5%, quite especially less than about 1% of the total soluble endogenous protein-bound N-glycans, or is below the detection limit of current analytical methods such as Western blot analysis using xylose-specific antibodies as described e.g. by Faye et al. (Analytical Biochemistry (1993) 209: 104-108) or such as mass spectrometry analysis of glycans isolated from the plant's glycoproteins using Matrix-Assisted Laser Desorption/Ionization Time-of-Flight Mass Spectrometry (MALDI-TOF-MS) as described e.g. by Kolarich and Altmann (Anal. Biochem. (2000) 285: 64-75), or using Liquid-Chromatography-ElectroSpray Ionization-Mass Spectrometry (LC/ESI/MS) as described by Pabst et al. (Analytical Chemistry (2007) 79: 5051-5057). Therefore, in the sense of the invention, a plant having a low level of β-1,2-xylose residues on protein-bound N-glycans is a plant in which the fraction of protein-bound N-glycans having β-1,2-xylose residues represents less than from 40 to 60%, especially less than from 20 to 40%, especially less than from 10 to 30%, especially less than from 10 to 20%, more especially less than from 5 to 15%; still more especially less than from 2 to 10%, quite especially less than from 0.1 to 2% of the total soluble endogenous protein-bound N-glycans, or is not detectable by current analytical methods. When the expression “a low level of β-1,2-xylose residues on protein-bound N-glycans” is used to qualify a foreign glycoprotein of interest, the above definition also applies but, in that case, refers to the N-glycans bound to the total foreign glycoproteins of interest and not to the total soluble endogenous proteins. Therefore, such a plant having a low level of β-1,2-xylose residues on protein-bound N-glycans may even have no detectable β-1,2-xylose residues on foreign glycoproteins, such as an antibody.

Similarly, “a plant having a low level of α-1,3-fucose residues” on protein-bound N-glycans is a plant (particularly a Nicotiana plant or a Nicotiana benthamiana plant), in which the α-1,3-fucosyltransferase activity is decreased in comparison to a control plant, resulting in a lower level of α-1,3-fucose residues in comparison to the level of α-1,3-fucose residues on protein-bound N-glycans of the control plant. The “control” plant is generally a selected target plant which could be used as a biofactory for producing therapeutic glycoproteins. Although such a “control” plant may be any plant, it may advantageously be selected among tobacco and related species like Nicotiana, including N. benthamiana, N. tabacum, and S. tuberosum, or other plants such as M. sativa. Generally, the control plant is an unmodified plant that has not been provided either with a silencing nucleic acid molecule targeted to the endogenous α-1,3-fucosyltransferase encoding gene (“FucT” gene) or with a FucT allele associated with a low level of α-1,3-fucosyltransferase activity. A plant having a “low level” of α-1,3-fucose residues on protein-bound N-glycans is a plant in which the fraction of protein-bound N-glycans having α-1,3-fucose residues represents less than about 50%, especially less that about 30%, especially less than about 20%, especially less than about 15%, more especially less than about 10%; still more especially less than about 5%, quite especially less than about 1% of the total soluble endogenous protein-bound N-glycans, or is below the detection limit of current analytical methods such as Western blot analysis using fucose-specific antibodies as described e.g. by Faye et al. (Analytical Biochemistry (1993) 2 09: 104-108) or such as mass spectrometry analysis of glycans isolated from the plant's glycoproteins using Matrix-Assisted Laser Desorption/Ionization Time-of-Flight Mass Spectrometry (MALDI-TOF-MS) as described e.g. by Kolarich and Altmann (Anal. Biochem. (2000) 285: 64-75) or using Liquid-Chromatography-ElectroSpray Ionization-Mass Spectrometry (LC/ESI/MS) as described by Pabst et al. (Analytical Chemistry (2007) 79: 5051-5057). Therefore, in the sense of the invention, a “plant having a low level of α-1,3-fucose residues” on protein-bound N-glycans is a plant in which the fraction of protein-bound N-glycans having α-1,3-fucose residues represents less than from 40 to 60%, especially less than from about 20 to 40%, especially less than from 10 to 30%, especially less than from 10 to 20%, more especially less than from 5 to 15%; still more especially less than from 2 to 10%, quite especially less than from 0.1 to 2% of the total soluble endogenous protein-bound N-glycans, or is not detectable by current analytical methods. When the expression “a low level of α-1,3-fucose residues on protein-bound N-glycans” is used to qualify a foreign glycoprotein of interest, the above definition also applies but, in that case, refers to the N-glycans bound to the total foreign glycoproteins of interest and not to the total soluble endogenous proteins. Such a plant having a low level of α-1,3-fucose residues on protein-bound N-glycans may even have no detectable core α-1,3-fucose residues on foreign glycoproteins, such as an antibody.

Similarly, “a plant having a low level of β-1,2-xylose residues and α-1,3-fucose residues” on protein-bound N-glycans is a plant having both a low level of β-1,2-xylose residues and a low level of α-1,3-fucose residues on protein-bound N-glycans, as defined above. Such a plant may even have no detectable β-1,2-xylose residues and no detectable core α-1,3-fucose residues on foreign glycoproteins, such as an antibody.

The term “gene” means a DNA sequence comprising a region (transcribed region), which is transcribed into an RNA molecule (e.g. a pre-mRNA, comprising intron sequences, which is then spliced into a mature mRNA) in a cell, operable linked to regulatory regions (e.g. a promoter). A gene may thus comprise several operably linked sequences, such as a promoter, a 5′ leader sequence comprising e.g. sequences involved in translation initiation, a (protein) coding region (cDNA or genomic DNA) and a 3′ non-translated sequence comprising e.g. transcription termination sites.

“Endogenous gene” is used to differentiate from a “foreign gene”, “transgene” or “chimeric gene”, and refers to a gene from a plant of a certain plant genus, species or variety, which has not been introduced into that plant by transformation (i.e. it is not a ‘transgene’), but which is normally present in plants of that genus, species or variety, or which is introduced in that plant from plants of another plant genus, species or variety, in which it is normally present, by normal breeding techniques or by somatic hybridization, e.g., by protoplast fusion. Similarly, an “endogenous protein” is encoded by an “endogenous gene” that has not been introduced into a plant or plant tissue by plant transformation. By opposition, an “exogenous gene” or “foreign gene” refers to a gene which is not normally present in plants of that genus, species or variety, and which has been introduced into that plant by transformation. Such an exogenous gene encodes a foreign protein.

The β-1,2-xylosyltransferase activity and the α-1,3-fucosyltransferase activity can be evaluated by determining the level of β-1,2-xylose residues and the level of α-1,3-fucose residues on protein-bound N-glycans from a plant, respectively. The level of β-1,2-xylose residues and the level of α-1,3-fucose residues on protein-bound N-glycans from a plant can be measured e.g. by Western blot analysis using xylose-specific antibodies and fucose-specific antibodies, respectively, as described e.g. by Faye et al. (Analytical Biochemistry (1993) 209: 104-108) or by mass spectrometry on glycans isolated from the plant's glycoproteins using Matrix-Assisted Laser Desorption/Ionization Time-of-Flight Mass Spectrometry (MALDI-TOF-MS) as described e.g. by Kolarich and Altmann (Anal. Biochem. (2000) 285: 64-75), or using Liquid-Chromatography-ElectroSpray Ionization-Mass Spectrometry (LC/ESI/MS) as described by Pabst et al. (Analytical Chemistry (2007) 79: 5051-5057) or using Liquid Chromatography Tandem Mass Spectrometry (LC/MS/MS) as described e.g. by Henriksson et al. (Biochem. J. (2003) 375: 61-73).

In one embodiment, the plant having a low level of β-1,2-xylose residues on protein-bound N-glycans is obtained by transcriptional or post-transcriptional silencing of the expression of the endogenous β-1,2-xylosyltransferase encoding gene(s) (“XylT” gene(s)) and, similarly, the plant having a low level of core α-1,3-fucose residues on protein-bound N-glycans is obtained by transcriptional or post-transcriptional silencing of the expression of the endogenous α-1,3-fucosyltransferase encoding gene(s) (“FucT” gene(s)). According to one aspect of this embodiment, gene silencing is carried out by providing a silencing RNA molecule to a plant.

In the above-described embodiment of the present invention, it is clear that if the plant's genome comprises more than one gene encoding a β-1,2-xylosyltransferase, the expression of at least one, preferably all, of the endogenous genes encoding said β-1,2-xylosyltransferase may be silenced. Similarly, if the plant's genome comprises more than one gene encoding an α-1,3-fucosyltransferase, the expression of at least one, preferably all, of the endogenous genes encoding said α-1,3-fucosyltransferase may be silenced.

As used herein, “silencing RNA” or “silencing RNA molecule” refers to any RNA molecule, which upon introduction into a plant cell, reduces the expression of a target gene. Such silencing RNA may e.g. be so-called “antisense RNA”, whereby the RNA molecule comprises a sequence of at least 20 consecutive nucleotides having at least 95% sequence identity to the complement of the sequence of the target nucleic acid, preferably the coding sequence of the target gene. However, antisense RNA may also be directed to regulatory sequences of target genes, including the promoter sequences and transcription termination and polyadenylation signals. Silencing RNA further includes so-called “sense RNA” whereby the RNA molecule comprises a sequence of at least 20 consecutive nucleotides having at least 95% sequence identity to the sequence of the target nucleic acid. Other silencing RNA may be “unpolyadenylated RNA” comprising at least 20 consecutive nucleotides having at least 95% sequence identity to the complement of the sequence of the target nucleic acid, such as described in WO01/12824 or U.S. Pat. No. 6,423,885 (both documents herein incorporated by reference). Yet another type of silencing RNA is an RNA molecule as described in WO03/076619 (herein incorporated by reference) comprising at least 20 consecutive nucleotides having at least 95% sequence identity to the sequence of the target nucleic acid or the complement thereof, and further comprising a largely-double stranded region as described in WO03/076619 (including largely double stranded regions comprising a nuclear localization signal from a viroid of the Potato spindle tuber viroid-type or comprising CUG trinucleotide repeats). Silencing RNA may also be double stranded RNA comprising a sense and antisense strands as herein defined, wherein the sense and antisense strands are capable of base-pairing with each other to form a double stranded RNA region (preferably the said at least 20 consecutive nucleotides of the sense and antisense RNA are complementary to each other). The sense and antisense regions may also be present within one RNA molecule such that a hairpin RNA (hpRNA) can be formed when the sense and antisense regions form a double stranded RNA region. hpRNA is well-known within the art (see e.g WO99/53050, herein incorporated by reference). The hpRNA may be classified as long hpRNA, having long, sense and antisense regions which can be largely complementary, but need not be entirely complementary (typically larger than about 200 bp, ranging between 200-1000 bp). hpRNA can also be rather small, ranging in size from about 30 to about 42 bp, but not much longer than 94 by (see WO04/073390, herein incorporated by reference). Silencing RNA may also be artificial micro-RNA molecules as described e.g. in WO05/052170, WO05/047505 or US 2005/0144667 (all documents incorporated herein by reference).

In one embodiment, the silencing RNA molecules are introduced in the plant or plant cell in the form of RNA. Methods for introducing RNA into plants are well known in the art and include infection with a suitable plant RNA virus comprising the desired RNA (Robertson Annual Review of Plant Biology (2004) 55: 495-519; U.S. Pat. No. 5,500,360).

In another embodiment, a chimeric gene is introduced in a plant or plant cell so as to produce a silencing RNA molecule within said plant cell.

Therefore, in one embodiment, the plant having a low level of β-1,2-xylose residues on protein-bound N-glycans is obtained by producing a transgenic plant cell or plant comprising a chimeric gene capable of producing a silencing RNA molecule, particularly a double stranded RNA (“dsRNA”) molecule, wherein the complementary RNA strand of such a dsRNA molecule comprises a part of a nucleotide sequence encoding a XylT protein, preferably obtainable from the same species or cultivar as the plant cells into which said chimeric gene is to be introduced, or wherein the complementary RNA strand of such a dsRNA molecule comprises a part of the nucleotide sequence of a XylT gene or a XylT cDNA, preferably obtainable from the same species or cultivar as the plant cells into which said chimeric gene is to be introduced; and, similarly, the plant having a low level of core α-1,3-fucose residues on protein-bound N-glycans is obtained by producing a transgenic plant cell or plant comprising a chimeric gene capable of producing a silencing RNA molecule, particularly a double stranded RNA (“dsRNA”) molecule, wherein the complementary RNA strand of such a dsRNA molecule comprises a part of a nucleotide sequence encoding a FucT protein, preferably obtainable from the same species or cultivar as the plant cells into which said chimeric gene is to be introduced, or wherein the complementary RNA strand of such a dsRNA molecule comprises a part of the nucleotide sequence of a FucT gene or a FucT cDNA, preferably obtainable from the same species or cultivar as the plant cells into which said chimeric gene is to be introduced.

The part of the nucleotide sequence encoding a XylT protein and the part of the nucleotide sequence of a XylT gene or a XylT cDNA, which are comprised within the silencing RNA molecule, particularly within one strand of the double stranded RNA molecule, should be at least 18 nucleotides long, but may vary from about 18 nucleotides (nt) up to a length equaling the length (in nucleotides) of the XylT protein-encoding sequence or the XylT gene or cDNA sequence. The total length of the sense or antisense nucleotide sequence may thus be at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least about 50 nucleotides, at least about 100 nucleotides, at least about 150 nucleotides, at least about 200 nucleotides, or at least about 500 nucleotides. It is expected that there is no upper limit to the total length of the sense or the antisense nucleotide sequence. However for practical reason (such as e.g. stability of the chimeric genes) it is expected that the length of the sense or antisense nucleotide sequence should not exceed 5000 nucleotides, particularly should not exceed 2500 nucleotides and could be limited to about 1000 nucleotides.

It will be appreciated that the longer the total length of the part of the nucleotide sequence encoding a XylT protein or the part of the nucleotide sequence of a XylT gene or a XylT cDNA (sense or antisense region) (said nucleotide sequences being later referred as “nucleic acid of interest”) is, the less stringent the requirements for sequence identity between these regions and the corresponding sequence in the endogenous XylT gene from the plant it complements are. Preferably, the nucleic acid of interest should have a sequence identity of at least about 75% with the corresponding target sequence, particularly at least about 80%, more particularly at least about 85%, quite particularly about 90%, especially about 95%, more especially about 100%, quite especially be identical to the corresponding part of the target sequence or its complement. However, it is preferred that the nucleic acid of interest always includes a sequence of about 18 consecutive nucleotides, particularly 18 consecutive nucleotides, 19 consecutive nucleotides, 20 consecutive nucleotides, 21 consecutive nucleotides, 22 consecutive nucleotides, 23 consecutive nucleotides, or 24 consecutive nucleotides, particularly about 25 consecutive nucleotides, more particularly about 50 nucleotides, especially about 100 nucleotides, quite especially about 150 nucleotides with 100% sequence identity to the corresponding part of the target XylT nucleic acid.

It is clear that the above statements regarding the length of the part of the nucleotide sequence comprised within the silencing RNA molecule described for silencing a XylT gene, similarly apply to the part of the nucleotide sequence encoding a FucT protein or the part of the nucleotide sequence of a FucT gene or a FucT cDNA.

“Stringent hybridization conditions” as used herein means that hybridization will generally occur if there is at least 95% and preferably at least 97% sequence identity between the probe and the target sequence. Examples of stringent hybridization conditions are overnight incubation in a solution comprising 50% formamide, 5×SSC (150 mM NaCl, 15 mM trisodium citrate), 50 mM sodium phosphate (pH 7.6), 5×Denhardt's solution, 10% dextran sulfate, and 20 μg/ml denatured, sheared carrier DNA such as salmon sperm DNA, followed by washing the hybridization support in 0.1×SSC at approximately 65° C., e.g. for about 10 min (twice). Other hybridization and wash conditions are well known and are exemplified in Sambrook et al., Molecular Cloning: A Laboratory Manual, Second Edition, Cold Spring Harbor, N.Y. (1989), particularly chapter 11.

For the purpose of this invention, the “sequence identity” of two related nucleotide or amino acid sequences, expressed as a percentage, refers to the number of positions in the two optimally aligned sequences which have identical residues (×100) divided by the number of positions compared. A gap, i.e., a position in an alignment where a residue is present in one sequence but not in the other is regarded as a position with non-identical residues. The alignment of the two sequences is performed by the Needleman and Wunsch algorithm (Needleman and Wunsch (1970) J. Mol. Biol. 48(3): 443-53). The computer-assisted sequence alignment above, can be conveniently performed using standard software program such as GAP which is part of the Wisconsin Package Version 10.1 (Genetics Computer Group, Madison, Wis., USA) using the default scoring matrix with a gap creation penalty of 50 and a gap extension penalty of 3. Sequences are indicated as “essentially similar” when such sequences have a sequence identity of at least about 75%, particularly at least about 80%, more particularly at least about 85%, quite particularly about 90%, especially about 95%, more especially about 100%, quite especially are identical. It is clear than when RNA sequences are said to be essentially similar or have a certain degree of sequence identity with DNA sequences, thymine (T) in the DNA sequence is considered equal to uracil (U) in the RNA sequence. Whether reference is made to RNA or DNA molecules will be clear from the context of the application.

It has been demonstrated that the minimum requirement for silencing a particular target gene is the presence in the silencing chimeric gene's nucleotide sequence of a nucleotide sequence of about 20 to 21 consecutive nucleotides long corresponding to the target gene sequence, in which at least 18 out of the 20-21 consecutive nucleotides are identical to the corresponding target gene sequence. “18 out of 21 consecutive nucleotides” as used herein refers to a nucleotide sequence of 21 consecutive nucleotides selected from the target gene having three mismatch nucleotides. “At least 18 out of 20-21 consecutive nucleotides” includes the following two alternatives: at least 18 out of 20 consecutive nucleotides and at least 18 out of 21 consecutive nucleotides.

For silencing the endogenous XylT gene from a plant, it is preferred that the silencing chimeric gene's nucleotide sequence comprises at least 18 out of 20-21 consecutive nucleotides selected from a nucleotide sequence encoding a XylT protein, or the complement thereof, said nucleotide sequence preferably obtainable from the same species or cultivar as the plant cells into which the chimeric gene is to be introduced, or selected from a nucleotide sequence of a XylT gene or a XylT cDNA, or the complement thereof, said nucleotide sequence preferably obtainable from the same species or cultivar as the plant cells into which the chimeric gene is to be introduced.

For silencing the endogenous FucT gene from a plant, it is preferred that the silencing chimeric gene's nucleotide sequence comprises at least 18 out of 20-21 consecutive nucleotides selected from a nucleotide sequence encoding a FucT protein, or the complement thereof, said nucleotide sequence preferably obtainable from the same species or cultivar as the plant cells into which the chimeric gene is to be introduced, or selected from a nucleotide sequence of a FucT gene or a FucT cDNA, or the complement thereof, said nucleotide sequence preferably obtainable from the same species or cultivar as the plant cells into which the chimeric gene is to be introduced.

Still more preferably, for silencing the endogenous XylT or FucT gene, the silencing chimeric gene's nucleotide sequence comprises at least 18 out of 21 consecutive nucleotides selected from the above described nucleotide sequences.

It has been found that double stranded RNA molecules, such as the ones described above, are cleaved in plant cells into small RNA fragments of about 20 nucleotides, in particular of 21 and 22 nucleotides, which serve as guide sequence in the degradation of the corresponding mRNA (reviewed by Baulcombe (2004) Nature 431: 356-363; Brosnan et al. (2007) PNAS 104(37): 14741-14746). Some 24 nucleotide long dsRNA have also been identified, which also play a role in gene silencing (Brosnan et al. (2007) PNAS 104(37): 14741-14746).

Some about 20 to 25 nucleotide long dsRNA sequences are also generated in the course of conventional antisense RNA mediated silencing or sense RNA mediated silencing.

The mentioned antisense or sense nucleotide regions may thus be from about 20-21 nucleotides to about 5000 nucleotides long, such as 20 nucleotides, 21 nucleotides, 22 nucleotide, 24 nucleotides, 25 nucleotides, 30 nucleotides, 40 nucleotides, 50 nucleotides, 100 nucleotides, 150 nucleotides, 200 nucleotides, 300 nucleotides, 500 nucleotides, 1000 nucleotides, or even about 2000 nucleotides or larger in length. Moreover, it is not required for the purpose of the invention that the nucleotide sequence of the silencing RNA molecule, or the region of the chimeric gene encoding the silencing RNA molecule, is completely identical or complementary to the nucleotide sequence of the endogenous gene to which it is targeted (XylT gene or FucT gene). The longer the sequence, the less stringent the requirement for the overall sequence identity is. Thus, the sense or antisense regions may have an overall sequence identity of about 40% or 50% or 60% or 70% or 80% or 90% or 100% to the nucleotide sequence of the endogenous gene or the complement thereof. However, as mentioned, antisense or sense regions should preferably comprise a nucleotide sequence of 18, 19, 20, 21 or 22 consecutive nucleotides having about 100% sequence identity to the target nucleotide sequence (XylT or FucT nucleotide sequence). The stretch of about 100% sequence identity may be about 50, 75 or 100 nucleotides.

In one embodiment, the invention is drawn to a method for producing a plant cell or plant having a low level of β-1,2-xylose residues and core α-1,3-fucose residues on protein-bound N-glycans comprising the steps of:
1) Producing a first transformed plant having a low level of β-1,2-xylose residues on protein-bound N-glycans by the method comprising the steps of:
   a) providing one or more first double stranded RNA molecules to plant cells or to a plant, wherein the first double stranded RNA molecule(s) comprise two RNA strands, one RNA strand consisting essentially of an RNA nucleotide sequence of at least 18 out of 20-21 consecutive nucleotides selected from a nucleotide sequence encoding a XylT protein, or the complement thereof, said nucleotide sequence preferably obtainable from the same species or cultivar as the cells of the plant into which the first double stranded RNA molecule(s) is to be introduced, or selected from the nucleotide sequence of a XylT gene or a XylT cDNA, or the complement thereof, said nucleotide sequence preferably obtainable from the same species or cultivar as the plant cells into which said first double stranded RNA molecule(s) is to be introduced;
   b) identifying a transformed plant cell comprising said first double stranded RNA molecule(s) which has a lower level of β-1,2-xylose residues on protein-bound N-glycans than an untransformed plant cell;
   c) optionally, regenerating one or more transformed plant cells from step a) or b) to obtain transformed plants;
   d) identifying, from the transformed plants obtained in step a) or c), a transformed plant which has a lower level of β-1,2-xylose residues on protein-bound N-glycans than an untransformed plant; and
2) Producing a second transformed plant having a low level of core α-1,3-fucose residues on protein-bound N-glycans by the method comprising the steps of:
   a) providing one or more second double stranded RNA molecules to plant cells or to a plant, wherein the second double stranded RNA molecules comprise two RNA strands, one RNA strand consisting essentially of an RNA nucleotide sequence of at least 18 out of 20-21 consecutive nucleotides selected from a nucleotide sequence encoding a FucT protein, or the complement thereof, said nucleotide sequence preferably obtainable from the same species or cultivar as the cells of the plant into which the second double stranded RNA molecule(s) is to be introduced, or selected from the nucleotide sequence of a FucT gene or a FucT cDNA, or the complement thereof, said nucleotide sequence preferably obtainable from the same species or cultivar as the plant cells into which said second double stranded RNA molecule(s) is to be introduced;
   b) optionally, identifying a transformed plant cell comprising said second double stranded RNA molecule(s) which has a lower level of core α-1,3-fucose residues on protein-bound N-glycans than an untransformed plant cell;
   c) optionally, regenerating one or more transformed plant cells from step a) or b) to obtain transformed plants;
   d) identifying, from the transformed plants obtained in step a) or c), a transformed plant which has a lower level of α-1,3-fucose residues on protein-bound N-glycans than an untransformed plant;
3) Crossing the first transformed plant of step 1) with the second transformed plant of step 2);
4) Optionally, identifying from the progeny obtained from the crossing of step 3) a transformed plant which has a low level of β-1,2-xylose and core α-1,3-fucose residues on protein-bound N-glycans.

According to one embodiment of the method of the invention, said first transformed plant having a low level of β-1,2-xylose residues on protein-bound N-glycans is produced by the method comprising the step of providing to plant cells a chimeric gene comprising, operably linked, the following DNA fragments: i) a plant expressible promoter; ii) a DNA region which, when transcribed, yields an RNA molecule capable of forming a double stranded RNA region at least between (1) an RNA region transcribed from a first sense DNA region comprising a nucleotide sequence of at least 18 out of 20-21 consecutive nucleotides selected from a nucleotide sequence encoding a XylT protein, or the complement thereof, said nucleotide sequence preferably obtainable from the same species or cultivar as the plant cells in the genome of which the first chimeric gene is to be integrated, or selected from a nucleotide sequence of a XylT gene or a XylT cDNA, or the complement thereof, said nucleotide sequence preferably obtainable from the same species or cultivar as the plant cells in the genome of which said first chimeric gene is to be integrated; and (2) an RNA region transcribed from a second antisense DNA region comprising a nucleotide sequence of at least 18 consecutive nucleotides which have at least 95% sequence identity to the complement of said first sense DNA region; and iii) a DNA region comprising a transcription termination and polyadenylation signal functional in plants; and said second transformed plant having a low level of core α-1,3-fucose residues on protein-bound N-glycans by the method comprising the step of providing to plant cells a chimeric gene comprising, operably linked, the following DNA fragments: i) a plant expressible promoter; ii) a DNA region which, when transcribed, yields an RNA molecule capable of forming a double stranded RNA region at least between (1) an RNA region transcribed from a third sense DNA region comprising a nucleotide sequence of at least 18 out of 20-21 consecutive nucleotides selected from a nucleotide sequence encoding a FucT protein, or the complement thereof, said nucleotide sequence preferably obtainable from the same species or cultivar as the plant cells in the genome of which said second chimeric gene is to be integrated, or selected from a nucleotide sequence of a FucT gene or a FucT cDNA, or the complement thereof, said nucleotide sequence preferably obtainable from the same species or cultivar as the plant cells in the genome of which said second chimeric gene is to be integrated; and (2) an RNA region transcribed from a fourth antisense DNA region comprising a nucleotide sequence of at least 18 consecutive nucleotides which have at least 95% sequence identity to the complement of said third sense DNA region; and iii) a DNA region comprising a transcription termination and polyadenylation signal functional in plants.

According to another embodiment of the method of the invention, said first transformed plant having a low level of β-1,2-xylose residues on protein-bound N-glycans is produced by the method comprising the step of providing to plant cells a chimeric gene comprising, operably linked, the following DNA fragments: i) a plant expressible promoter; ii) a DNA region comprising at least 18 out of 20-21 consecutive nucleotides selected from a nucleotide sequence encoding a XylT protein, or the complement thereof, said nucleotide sequence preferably obtainable from the same species or cultivar as the plant cells in the genome of which said chimeric gene is to be integrated, or selected from the nucleotide sequence of a XylT gene or a XylT cDNA, or the complement thereof, said nucleotide sequence preferably obtainable from the same species or cultivar as the plant cells in the genome of which said chimeric gene is to be integrated, in antisense or sense orientation; and iii) a DNA region comprising a transcription termination and polyadenylation signal functional in plants; and said second transformed plant having a low level of core α-1,3-fucose residues on protein-bound N-glycans is produced by the method comprising the step of providing to plant cells a chimeric gene comprising, operably linked, the following DNA fragments: i) a plant expressible promoter; ii) a DNA region comprising at least 18 out of 20-21 consecutive nucleotides selected from a nucleotide sequence encoding a FucT protein, or the complement thereof, said nucleotide sequence preferably obtainable from the same species or cultivar as the plant cells in the genome of which said chimeric gene is to be integrated, or selected from the nucleotide sequence of a FucT gene or a FucT cDNA, or the complement thereof, said nucleotide sequence preferably obtainable from the same species or cultivar as the plant cells in the genome of which said chimeric gene is to be integrated, in antisense or sense orientation; and iii) a DNA region comprising a transcription termination and polyadenylation signal functional in plants.

The chimeric genes according to the invention which encode dsRNA reducing the expression of XylT gene and the chimeric genes according to the invention which encode dsRNA reducing the expression of FucT gene may comprise an intron, such as a heterologous intron, located e.g. in the spacer sequence between the sense and antisense RNA regions in accordance with the disclosure of WO 99/53050 (incorporated herein by reference) or intron 2 from the A. thaliana XylT gene, which can be isolated as described in Example 2a) of the present application.

The efficiency of the above mentioned chimeric genes which when transcribed yield antisense or sense silencing RNA may be further enhanced by inclusion of DNA elements which result in the expression of aberrant, unpolyadenylated XylT (or FucT) inhibitory RNA molecules. One such DNA element suitable for that purpose is a DNA region encoding a self-splicing ribozyme, as described in WO00/01133. The efficiency may also be enhanced by providing the generated RNA molecules with nuclear localization or retention signals as described in WO03/076619.

Methods for the introduction of chimeric genes into plants are well known in the art and include Agrobacterium-mediated transformation, particle gun delivery, microinjection, electroporation of intact cells, polyethyleneglycol-mediated protoplast transformation, electroporation of protoplasts, liposome-mediated transformation, silicon-whiskers mediated transformation, etc. The transformed cells obtained in this way may then be regenerated into mature fertile plants.

In the sense of the invention, a XylT gene or a XylT cDNA from a plant refers to a nucleotide sequence of a XylT gene that naturally occurs in said plant or to cDNA corresponding to the mRNA of a XylT gene that naturally occurs in said plant. Similarly, a XylT protein from a plant refers to a protein as it naturally occurs in said plant.

Similarly, in the sense of the invention, a FucT gene or a FucT cDNA from a plant refers to a nucleotide sequence of a FucT gene that naturally occurs in said plant or to cDNA corresponding to the mRNA of a FucT gene that naturally occurs in said plant. Similarly, a FucT protein from a plant refers to a protein as it naturally occurs in said plant.

Examples of nucleotide sequences encoding a Nicotiana XylT protein, include those isolated from Nicotiana benthamiana encoding the amino acid sequence set forth in SEQ ID NO.: 10.

Examples of nucleotide sequences of a Nicotiana XylT gene include those isolated from Nicotiana benthamiana comprising the nucleotide sequence set forth in SEQ ID NO.: 9, as well as the prior art nucleotide sequences of XylT genes or cDNA isolated from other Nicotiana species such as the nucleotide sequence from Nicotiana tabacum cv. Xanthi available under accession numbers AJ627182 and AJ627183.

However, it will be immediately clear to the person skilled in the art that the exemplified nucleotide sequences or parts thereof can be used to identify further nucleotide sequences of Nicotiana XylT genes or Nicotiana XylT cDNAs in Nicotiana species or cultivars, and that such nucleotide sequences or parts thereof may also be used e.g. to decrease the level of β-1,2-xylose residues on protein-bound N-glycans in Nicotiana plants.

Examples of nucleotide sequences encoding a Nicotiana FucT protein, include those isolated from Nicotiana benthamiana encoding the amino acid sequence set forth in SEQ ID NO.: 27.

Examples of nucleotide sequences of a Nicotiana FucT gene include those isolated from Nicotiana benthamiana comprising the nucleotide sequence set forth in SEQ ID NO.: 26, as well as the nucleotide sequences of FucT genes or cDNA isolated from other Nicotiana species.

Similarly, it will be immediately clear to the person skilled in the art that the exemplified nucleotide sequences or parts thereof can be used to identify further nucleotide sequences of Nicotiana FucT genes or Nicotiana FucT cDNAs in Nicotiana species or cultivars, and that such nucleotide sequences or parts thereof may also be used e.g. to decrease the level of α-1,3-fucose residues on protein-bound N-glycans in Nicotiana plants.

The following DNA fragments or oligonucleotides could be used to identify and/or isolate FucT gene or cDNA of different Nicotiana species or cultivar, or new alleles of a given FucT gene:
i) a DNA fragment comprising a nucleotide sequence encoding the amino acid sequence of SEQ ID NO.: 27, for use as a probe;
ii) a DNA fragment comprising the nucleotide sequence of SEQ ID NO.: 26, for use as a probe;
iii) a DNA fragment or oligonucleotide comprising a nucleotide sequence consisting of between 20 to 200 consecutive nucleotides selected from a nucleotide sequence encoding the amino acid sequence of SEQ ID NO.: 27, for use as a probe;
iv) a DNA fragment or oligonucleotide comprising a nucleotide sequence consisting of between 20 to 1503 consecutive nucleotides selected from a nucleotide sequence encoding the amino acid sequence of SEQ ID NO.: 27, for use as a probe;
v) a DNA fragment or oligonucleotide comprising a nucleotide sequence consisting of between 20 to 200 consecutive nucleotides selected from a nucleotide sequence of SEQ ID NO.: 26, for use as a probe;
vi) a DNA fragment or oligonucleotide comprising a nucleotide sequence consisting of between 20 to 1503 consecutive nucleotides selected from a nucleotide sequence of SEQ ID NO.: 26, for use as a probe;
vii) an oligonucleotide having a nucleotide sequence comprising between 20 to 200 consecutive nucleotides selected from a nucleotide sequence encoding the amino acid sequence of SEQ ID NO.: 27, for use as a primer in a PCR reaction;
viii) an oligonucleotide having a nucleotide sequence comprising between 20 to 200 consecutive nucleotides selected from the nucleotide sequence of SEQ ID NO.: 26, for use as a primer in a PCR reaction; or
ix) an oligonucleotide having the nucleotide sequence of any one of SEQ ID NO.: 28 and SEQ ID NO.: 29, for use as a primer in a PCR reaction.

In the above-described method to identify and/or isolate a Nicotiana FucT gene or cDNA, or new allele of a FucT gene, it is preferred that said DNA fragment or oligonucleotide comprises at least one Nicotiana-specific FucT nucleotide and/or encodes at least one Nicotiana-specific FucT amino acid.

A “Nicotiana-specific FucT nucleotide” or a “Nicotiana-specific FucT nucleotide”, refers to a nucleotide of the nucleotide sequence of a FucT gene or a FucT cDNA from a Nicotiana species that differs from or is not present in the corresponding nucleotide sequence of the FucT gene from Arabidopsis thaliana (accession numbers AJ345084; AJ345085, NM112815, NM103858, At1g49710, At3g19280, AJ345084, AJ345085, AF154111, NM106102), Hordeum vulgare (AJ582181), Lemna minor (DQ789145), Medicago sativa (AY082444; AY082445), Medicago truncatula (AY557602), Oryza sativa (AK099681), Physcomitrella patens (AJ618932, AJ429145), Populus alba×Populus tremula (AJ891040), Triticum aestivum (AJ582182), Vigna radiata (Y18529, CAB52254) and Zea mays (AY964641).

A “Nicotiana-specific FucT amino acid” or a “Nicotiana-specific FucT amino acid”, refers to an amino acid of the amino acid sequence of a FucT protein encoded by a FucT gene or encoded by a FucT cDNA from a Nicotiana species that differs from or is not present in the corresponding amino acid sequence of the FucT protein encoded by the FucT gene from Arabidopsis thaliana (accession number CAC78979, CAC78980), Hordeum vulgare (CAE46648), Lemna minor (ABG89268), Medicago sativa (AAL99370; AAL99371), Medicago truncatula (AAS66306.1), Oryza sativa (BAD09365), Physomitrella patens (Q6A2M3, Q8L5D1), Populus alba×Populus tremula (CAI70373), Triticum aestivum (CAE46649), Vigna radiata (Q9ST51), and Zea mays (Q0VH31).

To determine the presence of a Nicotiana-specific FucT nucleotide or amino acid in the nucleotide sequence of a FucT gene or a FucT cDNA from a Nicotiana species or in the amino acid sequence of a FucT protein encoded by a FucT gene or encoded by a FucT cDNA from a Nicotiana species, for the purpose of this invention, the FucT nucleotide sequence or FucT amino acid sequence from the Nicotiana species is compared with the corresponding FucT nucleotide sequence or amino acid sequence from Arabidopsis thaliana, Hordeum vulgare, Lemna minor, Medicago sativa, Medicago trunculata, Oryza sativa, Physcomitrella patens, Populus alba×Populus tremula, Triticum aestivum, Vigna radiata or Zea mays by aligning the sequences indicated above in using a global alignment procedure (For nucleotide sequences the default scoring matrix used is “standard linear” with mismatch penalty=2, open gap penalty=4 and extend gap penalty=1. For protein sequences the default scoring matrix is “blosum 62”; Henikoff and Henikoff, 1992.). To perform the alignment the Align Plus program (provided by Scientific & Educational Software, USA) may be used.

Thus, by performing a PCR reaction using genomic DNA or cDNA from Nicotiana species or cultivars and the above-mentioned oligonucleotides as primers or by performing hybridization, preferably under stringent conditions between genomic or cDNA from Nicotiana species or cultivars and the above-mentioned probes, novel Nicotiana FucT genes or Nicotiana FucT cDNAs or fragments thereof can be identified and/or isolated.

The exemplified FucT nucleotide sequences from Nicotiana benthamiana can also be used to identify FucT alleles in a population of plants of a Nicotiana species or cultivar which are correlated with low levels of α-1,3-fucose residues on protein-bound N-glycans. Such populations of plants of a Nicotiana species or cultivar may be populations which have been previously mutagenized. The identified FucT alleles may then be introduced into a plant line of a Nicotiana species or cultivar of choice using conventional breeding techniques.

Therefore, another object of the invention relates to a method to obtain a plant cell or plant with a low level of β-1,2-xylose residues and α-1,3-fucose residues on protein-bound N-glycans comprising the steps of:
providing a first plant wherein the XylT activity has been reduced by deleting, disrupting, or replacing the endogenous XylT gene(s) and integrating, in said first plant, an exogenous XylT allele correlated with a low level of β-1,2-xylose residues on protein-bound N-glycans; and
providing a second plant wherein the FucT activity has been reduced by deleting, disrupting, or replacing the endogenous FucT gene(s) and integrating, in said second plant, an exogenous FucT allele correlated with a low level of core α-1,3-fucose residues on protein-bound N-glycans; and
crossing said first and second plants.

In the above-described object of the invention, it is clear that if the plant's genome comprises more than one gene encoding a β-1,2-xylosyltransferase, the expression of at least one, preferably all, of the endogenous genes encoding said β-1,2-xylosyltransferase may be deleted, disrupted or replaced. Similarly, if the plant's genome comprises more than one gene encoding an α-1,3-fucosyltransferase, the expression of at least one, preferably all, of the endogenous genes encoding said α-1,3-fucosyltransferase may be deleted, disrupted or replaced.

The present invention also concerns a method to obtain a Nicotiana plant cell or plant with a low level of α-1,3-fucose residues on protein-bound N-glycans, comprising the steps of: (i) identifying a Nicotiana FucT allele correlated with a low level of α-1,3-fucose residues on protein-bound N-glycans; (ii) introducing said Nicotiana FucT allele into a second plant of a Nicotiana plant line of choice; and (iii) optionally, identifying a Nicotiana plant, such as a transgenic Nicotiana plant, which has a lower level of α-1,3-fucose residues on protein-bound N-glycans than an untransformed Nicotiana plant.

The present invention more particularly concerns a method to obtain a Nicotiana plant cell or plant with a low level of β-1,2-xylose residues and α-1,3-fucose residues on protein-bound N-glycans, comprising the steps of:
a) identifying a Nicotiana XylT allele correlated with a low level of β-1,2-xylose residues on protein-bound N-glycans and introducing said Nicotiana XylT allele into a first plant of a Nicotiana plant line of choice;
b) identifying a Nicotiana FucT allele correlated with a low level of α-1,3-fucose residues on protein-bound N-glycans and introducing said Nicotiana FucT allele into a second plant of a Nicotiana plant line of choice; wherein the plant line from which said second plant originates can be the same or not as the plant line from which said first plant originates;
c) crossing a transgenic plant obtained in step a) with a transgenic plant obtained in step b) to obtain transgenic Nicotiana plants;
d) optionally, identifying a transgenic Nicotiana plant which has a lower level of β-1,2-xylose residues and α-1,3-fucose residues on protein-bound N-glycans than an untransformed Nicotiana plant.

The plant cell or plant having a low level of β-1,2-xylose residues and core α-1,3-fucose residues on protein-bound N-glycans produced according to the methods described in the present application are particularly useful as bioreactor for producing glycoproteins exhibiting an altered or modified N-glycans profile.

The alteration or modification of the N-glycans profile of the glycoproteins may result in altered functionality, folding or half-life of said glycoproteins.

Encompassed by the invention are glycoproteins which are endogenous to the plant cell as well as glycoproteins which are foreign to the cell of the plant, i.e. which are not normally expressed in such plant cells in nature. The foreign glycoproteins may include mammalian or human proteins, which can be used as therapeutics such as e.g. monoclonal antibodies, blood and plasma proteins, antigens for vaccination purposes, growth factors, hormones, cytokines, and enzymes with therapeutic potential. Conveniently, the foreign glycoproteins may be expressed from chimeric genes comprising a plant-expressible promoter and the coding region of the glycoprotein of interest, whereby the chimeric gene is stably integrated in the genome of the plant cell. Methods to express foreign proteins in plant cells are well known in the art. Alternatively, the foreign glycoproteins may also be expressed in a transient manner, e.g. using the viral vectors and methods described in WO02/088369, WO06/079546 or WO06/012906 or using the viral vectors described in WO89/08145, WO93/03161 or WO96/40867 or WO96/12028.

Thus, another embodiment of the present invention relates to the use of a plant obtained according to any method according to the invention described above for producing a foreign glycoprotein of interest having a low level of, or no detectable, β-1,2-xylose and α-1,3-fucose residues on N-glycans bound to said foreign glycoprotein.

Thus, also encompassed by the invention is a method to produce a foreign glycoprotein of interest having a low level of, or no detectable, β-1,2-xylose and α-1,3-fucose residues on N-glycans bound to said foreign glycoprotein, comprising:
1) producing a plant cell or plant having a low level of β-1,2-xylose residues and core α-1,3-fucose residues on protein-bound N-glycans by carrying out a method as described above;
2) providing to a plant cell or plant obtained in step 1) a chimeric gene comprising the following operably linked DNA fragments: a plant expressible promoter, a DNA region encoding the glycoprotein of interest, and a DNA region comprising a transcription termination and polyadenylation signal functional in plants;
3) optionally, identifying a transgenic plant or plant cell expressing the glycoprotein of interest;
4) cultivating the transgenic plant or plant cell obtained in step 3);
5) optionally, extracting and purifying the foreign glycoprotein of interest from the total plant proteins.

As used herein, the term “plant-expressible promoter” means a DNA sequence that is capable of controlling (initiating) transcription in a plant cell. This includes any promoter of plant origin, but also any promoter of non-plant origin which is capable of directing transcription in a plant cell, i.e., certain promoters of viral or bacterial origin such as the CaMV35S (Odell et al. (1985) Nature 313: 810; Hapster et al. (1988) Mol. Gen. Genet. 212, 182-190), the subterranean clover virus promoter No 4 or No 7 (WO9606932), or T-DNA gene promoters but also tissue-specific or organ-specific promoters including but not limited to seed-specific promoters (e.g., WO89/03887), organ-primordia specific promoters (An et al. (1996) Plant Cell 8: 15-30), stem-specific promoters (Keller et al. (1988) EMBO J. 7: 3625-3633), leaf specific promoters (Hudspeth et al. (1989) Plant Mol. Biol. 12: 579-589), mesophyl-specific promoters (such as the light-inducible Rubisco promoters), root-specific promoters (Keller et al. (1989) Genes Devel. 3: 1639-1646), tuber-specific promoters (Keil et al. (1989) EMBO J. 8: 1323-1330), vascular tissue specific promoters (Peleman et al. (1989) Gene 84: 359-369), stamen-selective promoters (WO 89/10396, WO 92/13956), dehiscence zone specific promoters (WO 97/13865) and the like.

Another embodiment of the invention relates to an isolated DNA fragment encoding a FucT protein comprising the nucleotide sequence of SEQ ID NO.: 26 or any part thereof comprising at least 20, at least 21, at least 25, at least 50, at least 100, at least 150, or at least 200, contiguous nucleotides, wherein said part preferentially comprises at least one Nicotiana-specific FucT nucleotide and/or encodes at least one Nicotiana-specific FucT amino acid.

Still another embodiment of the invention relates to a chimeric gene comprising the following operably linked DNA fragments:
a) a plant expressible promoter;
b) a DNA region which, when transcribed, yields an RNA molecule capable of forming a double stranded RNA region by base-pairing at least between:
   i) an RNA region transcribed from a first DNA region comprising at least 18 out of 20-21, at least 19, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in antisense orientation;
   ii) an RNA region transcribed from a second DNA region comprising at least 18 out of 20-21, at least 19, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in sense orientation; and
c) a DNA region comprising a transcription termination and polyadenylation signal functional in plants.

A still further embodiment of the invention relates to a chimeric gene comprising the following operably linked DNA fragments:
a) a plant expressible promoter;
b) a DNA region comprising at least 18 out of 20-21, at least 19, at least 20, at least 21, at least 22, at least 25, at least 50, at least 100, at least 150, or at least 200, consecutive nucleotides selected from a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in sense or antisense orientation; and
c) a DNA region comprising a transcription termination and polyadenylation signal functional in plants.

Also encompassed within the invention is a plant cell comprising:
1) a first chimeric gene capable of producing a silencing RNA molecule, particularly a double stranded RNA (“dsRNA”) molecule, wherein the complementary RNA strands of such a dsRNA molecule comprises a part of a nucleotide sequence encoding a XylT protein; and
2) a second chimeric gene capable of producing a silencing RNA molecule, particularly a double stranded RNA (“dsRNA”) molecule, wherein the complementary RNA strands of such a dsRNA molecule comprises a part of a nucleotide sequence encoding a FucT protein;
wherein said first and second chimeric genes are placed at unlinked positions in the genome of said plant cell.

A plant cell of the invention advantageously comprises the above-described chimeric genes.

Also encompassed by the invention is a plant obtained after regeneration of a plant cell according to the invention, as well as the seeds produced by said plant.

Gametes, seeds, embryos, progeny, hybrids of plants, or plant tissues including stems, leaves, stamen, ovaria, roots, meristems, flowers, seeds, fruits, fibers comprising the chimeric genes of the present invention, which are produced by traditional breeding methods are also included within the scope of the present invention.

The obtained plants having a low level of β-1,2-xylose residues and core α-1,3-fucose residues on protein-bound N-glycans according to the invention can be used in a conventional breeding scheme to produce more plants with the same characteristics or to introduce the chimeric genes according to the invention in other cultivars of the same or related plant species, or in hybrid plants. Seeds obtained from the transformed plants contain the chimeric genes of the invention as a stable genomic insert and are also encompassed by the invention.

Furthermore, it is known that introduction of antisense, sense or double-stranded RNA or the encoding chimeric genes may lead to a distribution of phenotypes, ranging from almost no or very little suppression of the expression of the target gene to a very strong or even a 100% suppression of the expression of the target gene. However, a person skilled in the art will be able to select those plant cells, plants, events or plant lines leading to the desired degree of silencing and desired phenotype.

The methods and means described herein are believed to be suitable for all plant cells and plants, gymnosperms and angiosperms, both dicotyledonous and monocotyledonous plant cells and plants including but not limited to Arabidopsis, alfalfa, barley, bean, corn or maize, cotton, flax, oat, pea, rape, rice, rye, safflower, sorghum, soybean, sunflower, tobacco and other Nicotiana species, including Nicotiana benthamiana, wheat, asparagus, beet, broccoli, cabbage, carrot, cauliflower, celery, cucumber, eggplant, lettuce, onion, oilseed rape, pepper, potato, pumpkin, radish, spinach, squash, tomato, zucchini, almond, apple, apricot, banana, blackberry, blueberry, cacao, cherry, coconut, cranberry, date, grape, grapefruit, guava, kiwi, lemon, lime, mango, melon, nectarine, orange, papaya, passion fruit, peach, peanut, pear, pineapple, pistachio, plum, raspberry, strawberry, tangerine, walnut and watermelon Brassica vegetables, sugarcane, vegetables (including chicory, lettuce, tomato) and sugarbeet.

In a particular embodiment the plants having a low level of β-1,2-xylose residues on protein-bound N-glycans and those having a low level of core α-1,3-fucose residues on protein-bound N-glycans are plants from any Nicotiana species or cultivar. In another embodiment said plants are Nicotiana benthamiana. In a further embodiment, both kinds of plants are from the same species or cultivar.

“Nicotiana”, as used herein, includes all known Nicotiana species, such as, but not limited to, Nicotiana acaulis, N. acuminata, N. africana, N. alata, N. amplexicaulis, N. arentsii, N. attenuata, N. benavidesii, N. benthamiana, N. bigelovii, N. bonariensis, N. cavicola, N. clevelandii, N. cordifolia, N. corymbosa, N. debneyi, N. excelsior, N. forgetiana, N. fragrans, N. glauca, N. glutinosa, N. goodspeedii, N. gossei, N. hybrid, N. ingulba, N. kawakamii, N. knightiana, N. langsdorffii, N. linearis, N. longiflora, N. maritima, N. megalosiphon, N. miersii, N. noctiflora, N. nudicaulis, N. obtusifolia, N. occidentalis, N. otophora, N. paniculata, N. pauciflora, N. petunioides, N. plumbaginifolia, N. quadrivalvis, N. raimondii, N. repanda, N. rosulata, N. rotundifolia, N. rustica, N. setchellii, N. simulans, N. solanifolia, N. spegazzinii, N. stocktonii, N. suaveolens, N. sylvestris, N. tabacum, N. thyrsiflora, N. tomentosa, N. tomentosiformis, N. trigonophylla, N. umbratica, N. undulata, N. velutina, N. wigandioides, and Nicotiana×sandera, and all known Nicotiana cultivars, such as, but not limited to, cultivars of Nicotiana tabacum, such as cv. Burley21, cv. Delgold, cv. Petit Havana, cv. Petit Havana SR1, cv. Samsun, and cv. Xanthi.

As used herein “comprising” is to be interpreted as specifying the presence of the stated features, integers, steps or components as referred to, but does not preclude the presence or addition of one or more features, integers, steps or components, or groups thereof. Thus, e.g., a nucleic acid or protein comprising a sequence of nucleotides or amino acids, may comprise more nucleotides or amino acids than the actually cited ones, i.e., be embedded in a larger nucleic acid or protein. A chimeric gene comprising a DNA region, which is functionally or structurally defined, may comprise additional DNA regions etc.

The following non-limiting Examples describe chimeric genes for the alteration of the level of β-1,2-xylose residues and α-1,3-fucose residues on protein-bound N-glycans in Nicotiana species, particularly in Nicotiana benthamiana, and uses thereof. These examples also demonstrate that a plant obtained according to the method of the invention produces human monoclonal antibodies with no detectable xylose and fucose residues. Unless stated otherwise in the Examples, all recombinant DNA techniques are carried out according to standard protocols as described in Sambrook et al. (1989) Molecular Cloning: A Laboratory Manual, Second Edition, Cold Spring Harbor Laboratory Press, NY and in Volumes 1 and 2 of Ausubel et al. (1994) Current Protocols in Molecular Biology, Current Protocols, USA. Standard materials and methods for plant molecular work are described in Plant Molecular Biology Labfax (1993) by R. D. D. Croy, jointly published by BIOS Scientific Publications Ltd (UK) and Blackwell Scientific Publications, UK.

Throughout the description and Examples, reference is made to the following sequences represented in the sequence listing:
SEQ ID NO: 1: nucleotide sequence of an oligonucleotide suitable to amplify a part of a Nicotiana benthamiana XylT gene or cDNA
SEQ ID NO: 2: nucleotide sequence of an oligonucleotide suitable to amplify a part of a Nicotiana benthamiana XylT gene or cDNA
SEQ ID NO: 3: nucleotide sequence of an oligonucleotide suitable to amplify the 3′-end of a Nicotiana benthamiana XylT gene or cDNA
SEQ ID NO: 4: nucleotide sequence of an oligonucleotide suitable to amplify the 3′-end of a Nicotiana benthamiana XylT gene or cDNA
SEQ ID NO: 5: nucleotide sequence of an oligonucleotide suitable to amplify the 3′-end of a Nicotiana benthamiana XylT gene or cDNA
SEQ ID NO: 6: nucleotide sequence of an oligonucleotide suitable to amplify the 3′-end of a Nicotiana benthamiana XylT gene or cDNA
SEQ ID NO: 7: nucleotide sequence of an oligonucleotide suitable to amplify a Nicotiana benthamiana XylT cDNA
SEQ ID NO: 8: nucleotide sequence of an oligonucleotide suitable to amplify a Nicotiana benthamiana XylT cDNA
SEQ ID NO: 9: nucleotide sequence of a Nicotiana benthamiana XylT cDNA
SEQ ID NO: 10: amino acid sequence of a Nicotiana benthamiana XylT protein
SEQ ID NO: 11: nucleotide sequence of an oligonucleotide suitable to amplify a part of Arabidopsis thaliana XylT gene
SEQ ID NO: 12: nucleotide sequence of an oligonucleotide suitable to amplify a part of Arabidopsis thaliana XylT gene
SEQ ID NO: 13: nucleotide sequence of an oligonucleotide suitable to amplify intron 2 of Arabidopsis thaliana XylT gene
SEQ ID NO: 14: nucleotide sequence of an oligonucleotide suitable to amplify intron 2 of Arabidopsis thaliana XylT gene
SEQ ID NO: 15: nucleotide sequence of the oligonucleotide NBXT25 suitable to amplify an antisense fragment of a Nicotiana benthamiana XylT gene
SEQ ID NO: 16: nucleotide sequence of the oligonucleotide NBXT26 suitable to amplify an antisense fragment of a Nicotiana benthamiana XylT gene
SEQ ID NO: 17: nucleotide sequence of a XylT-RNAi construct (pGAX1)
SEQ ID NO: 18: nucleotide sequence of the degenerated primer FTAD1 suitable to amplify a part of a Nicotiana benthamiana FucT gene or cDNA.
SEQ ID NO: 19: nucleotide sequence of the degenerated primer FTAD2 suitable to amplify a part of a Nicotiana benthamiana FucT gene or cDNA.
SEQ ID NO: 20: nucleotide sequence of an oligonucleotide suitable to amplify the 5′-end or 3′-end of a Nicotiana benthamiana FucT gene or cDNA
SEQ ID NO: 21: nucleotide sequence of an oligonucleotide suitable to amplify the 5′-end or 3′-end of a Nicotiana benthamiana FucT gene or cDNA
SEQ ID NO: 22: nucleotide sequence of an oligonucleotide suitable to amplify the 5′-end of a Nicotiana benthamiana FucT gene or cDNA
SEQ ID NO: 23: nucleotide sequence of an oligonucleotide suitable to amplify the 3′-end of a Nicotiana benthamiana FucT gene or cDNA
SEQ ID NO: 24: nucleotide sequence of an oligonucleotide suitable to amplify the 3′-end of a Nicotiana benthamiana FucT gene or cDNA
SEQ ID NO: 25: nucleotide sequence of an oligonucleotide suitable to amplify the 3′-end of a Nicotiana benthamiana FucT gene or cDNA
SEQ ID NO.: 26: nucleotide sequence of a Nicotiana benthamiana FucT cDNA
SEQ ID NO.: 27: amino acid sequence of a Nicotiana benthamiana FucT protein
SEQ ID NO.: 28: nucleotide sequence of the oligonucleotide NBFT1 suitable to amplify a sense fragment of a Nicotiana benthamiana FucT gene
SEQ ID NO.: 29: nucleotide sequence of the oligonucleotide NBFT2 suitable to amplify a sense or antisense fragment of a Nicotiana benthamiana FucT gene
SEQ ID NO.: 30: nucleotide sequence of the oligonucleotide NBFT2 suitable to amplify an antisense fragment of a Nicotiana benthamiana FucT gene
SEQ ID NO.: 31: nucleotide sequence of a FucT-RNAi construct (pGAX3)

### EXAMPLES

### Example 1

### Isolation of XylT cDNA Sequences from Nicotiana benthamiana

RNA was extracted from leaves of Nicotiana benthamiana using the TRIZOL® Reagent (Invitrogen Life Technologies) according to the manufacturer's protocol and used for cDNA synthesis using SuperScript™ First-strand synthesis System for RT-PCR (Invitrogen Life Technologies) according to the manufacturer's instructions.

Oligonucleotide sequences to be used as primers in a PCR amplification of XylT cDNA from Nicotiana benthamiana were designed based on Tomato EST clone coding for XylT (BG 130152). The following primers were generated:

Tom-XT21 (SEQ ID NO.: 1):
5′-GAGGATTATTTAGCTCACCCACG-3′
Tom-XT23 (SEQ ID NO.: 2):
5′-AGCAGCCAAGACTCCTCAAAAT-3′

Using the cDNA as template and the above-described primer pair, a PCR reaction was performed under the following conditions:

5 min at 95° C.; followed by 8 cycles comprising 15 sec at 94° C. (denaturation), 30 sec at 65° C. (annealing), 2 min at 72° C. (elongation); followed by 30 cycles comprising 15 sec at 94° C. (denaturation), 30 sec at 53° C. (annealing), 2 min at 72° C. (elongation); followed by a final extension step of 4 min at 72° C.

A DNA fragment (partial XylT cDNA) of about 364 basepairs was amplified, cloned into a pCR 2.1-TOPO® vector (Invitrogen) and 2 different clones were obtained (comprising the sequences of NbXT1 and NbXT2) yielding TOPO-XT1 and TOPO-XT2, respectively.

The 3′-end of the cDNA was isolated by carrying out a 3′-RACE PCR using a GeneRacer Kit (Invitrogen) according to the manufacturer's protocol. This 3′-RACE PCR comprised the two successive PCR reactions as follows:

a) a First PCR Using the Following Primers:

Forward primer: XT24 (SEQ ID NO.: 3):
5′-TATATGTCGACTCTAGATTAGCAATGAAGAGCAAGTA-3′
Reverse primer: GeneRacer ™
3′ primer (SEQ ID NO.: 4):
5′-GCTGTCAACGATACGCTACGTAACG-3′

b) a Second Nested PCR Using the Following Primers:

Forward primer: NbXT31 (SEQ ID NO.: 5):
5′-GGTGCTCATGGAGCAGGTCTAAC-3′
Reverse primer: GeneRacer ™ 3′ Nested primer
(SEQ ID NO.: 6):
5′-CGCTACGTAACGGCATGACAGTG-3′

Both first and second PCR were performed under the following conditions: 3 min at 94° C.; 5 cycles of 1 min at 94° C., 1 min at 55° C. and 1 min 30 sec at 72° C.; 30 cycles of 1 min at 94° C., 1 min at 50° C. and 1 min 30 sec at 72° C.; followed by 10 min at 72° C.

The PCR product was cloned into pCR2.1-TOPO® (Invitrogen) yielding one clone P1 and sequenced. When combined with the partial cDNA sequences NbXT1 and NbXT2 obtained as described above, this 3′-RACE PCR lead to the identification of two different 3′-XylT nucleotide sequences NbXT131 and NbXT231.

Finally, the “complete” cDNA sequence represented by NbXT131 was isolated by carrying out a RT-PCR reaction on N. benthamiana leaf cDNA using the following primers:

NBXT32 (SEQ ID NO.: 7): 5′-AGTCAGAGAGAGAAGAAGATG
AACAAGAA-3′
NBXT34 (SEQ ID NO.: 8): 5′-GAACTATTCAAACTGTCGA
GCGGA-3′

under the following conditions: 1 min at 95° C. and 40 cycles of 20 sec at 95° C., 20 sec at 55° C. and 2 min 20 sec at 68° C.

The sequences of both primers were based on the sequence of a Nicotiana tabacum mRNA for putative β-(1,2)-xylosyltransferase (accession number AJ627182).

After purification from an agarose gel, the PCR product was cloned into pCR4Blunt-TOPO® (Invitrogen) yielding clone pCR4-Nb-XT-1600.

This protocol allowed the identification of a XylT cDNA nucleotide sequence of 1551 by represented under SEQ ID NO.: 9.

This XylT cDNA nucleotide sequence encodes a protein of amino acid sequence SEQ ID NO.: 10.

The results of the comparison between the amino acid sequence of the putative XylT protein encoded by the cDNA sequence from Arabidopsis thaliana (accession number AJ272121), Lemna minor (DQ789144), Medicago sativa (AY302251), Oryza sativa (AP004190), Physcomitrella patens (PPA492144), Zea mays (DQ026518), and the amino acid sequence of XylT protein from Nicotiana benthamiana (SEQ ID NO.: 10) are presented in Table 2.

TABLE 2

Percentage of identity between the amino acid sequences
of XylT protein of different plants.

| | | | | | | |
|---|---|---|---|---|---|---|
| | | | | | | Nb XylT |
| | | | | | | SEQ ID |
| Ath XylT | Lm XylT | Php XylT | Os XylT | Zm XylT | Ms XylT | NO.: 10 |

| | | | | | | |
|---|---|---|---|---|---|---|
| Ath XylT | 53 | 42 | 57 | 56 | 62 | 64 |
| Lm XylT | | 41 | 56 | 55 | 55 | 53 |
| Php XylT | | | 42 | 43 | 41 | 40 |
| Os XylT | | | | 82 | 59 | 58 |
| Zm XylT | | | | | 57 | 56 |
| Ms XylT | | | | | | 64 |
| Nb XylT | | | | | | |
| SEQ ID | | | | | | |
| NO.: 10 | | | | | | |

Abbreviations:
Ath: Arabidopsis thaliana,
Lm: Lemna minor,
Php: Physcomitrella patens,
Os: Oryza sativa,
Zm: Zea mays,
Ms: Medicago sativa,
Nb: Nicotiana benthamiana

### Example 2

### Construction of a T-DNA Vector Containing a Nicotiana benthamiana XylT Silencing Gene (XylT-RNAi Construct)

DNA fragments amplified from Nicotiana benthamiana XylT sequences described in Example 1 were used to construct T-DNA vectors comprising a chimeric gene which upon transcription yields an RNA molecule comprising a sense and antisense XylT DNA sequence from the amplified DNA fragment, and which could basepair to form a double stranded RNA molecule. Such a chimeric gene can be used to reduce the expression of a XylT gene in Nicotiana, particularly in Nicotiana benthamiana.

a) Cloning of the Intron 2 from the A. thaliana XylT Gene

First, a XylT DNA fragment from the A. thaliana XylT gene (Accession Number At5g55500) was amplified by PCR using the genomic DNA from A. thaliana ecotype Wassilewskija WS-3 as template with the following oligonucleotides as primers:

Forward primer: XT1 (SEQ ID NO.: 11):
5′-ATTCTCGCTCTCTCTTCAAAACCGCAAAT-3′
Reverse primer: XT2 (SEQ ID NO.: 12):
5′-GTCACCGGAGATTAGAACTCACTCACTAT-3′

and the following PCR conditions: 5 min at 95° C.; followed by 38 cycles of 15 sec at 94° C., 30 sec at 65° C., 2 min at 72° C.; and a final extension step of 4 min at 72° C.

In a second PCR reaction, intron 2 from the A. thaliana XylT gene was amplified from the above-mentioned XylT DNA fragment used as a template, using the following primers:

Forward primer: ARA_XTI2fw (SEQ ID NO.: 13):
5′-ATCAGGGATCCACTGCACGGTATGCTCCTC-3′
Reverse primer: ARA_XTI2rv (SEQ ID NO.: 14):
5′-ATCGTGGTACCTAGCTGCGTCTGCAAAAAG-3′

and the following PCR conditions: 2 min at 95° C.; followed by 25 cycles of 45 sec at 56° C., 30 sec at 72° C., and 20 sec at 94° C.

The PCR product was purified, digested by BamHI and KpnI and ligated into BamHI/KpnI digested cloning vector puc18, leading to vector p18I2.

### b) Cloning of the Sense XylT Sequence

Oligonucleotide sequences to be used as non-degenerated primers in a PCR amplification of a XylT gene sequence from Nicotiana benthamiana were designed based on the cDNA sequence from Nicotiana benthamiana isolated as described above in Example 1.

The sense XylT fragment was produced by PCR using the vector Topo-XT-1 comprising a cDNA fragment amplified from Nicotiana benthamiana leaf mRNA as described above (NbXT1) as template and the following primers:

Forward primer: XT24 (SEQ ID NO.: 3):
5′-TATATGTCGACTCTAGATTAGCAATGAAGAGCAAGTA-3′
Reverse primer: TomXT23 (SEQ ID NO.: 2):
5′-AGCAGCCAAGACTCCTCAAAAT-3′

under the following conditions: 2 min at 95° C., followed by 25 cycles comprising: 45 sec at 55° C., 30 sec at 72° C. and 20 sec at 94° C.

The PCR product was purified, digested by SalI/BamHI (the XylT DNA fragment sequence contains an internal BamHI site) and cloned into SalI/BamHI digested cloning vector p18I2 to create p18Xsi.

### c) Cloning of the Antisense XylT Sequence

The antisense XylT fragment was produced by PCR also using Topo-XT-1 as template but with the following primers:

Forward primer: XT25 (SEQ ID NO.: 15):
5′-TATATGAATTCTAGATTAGCAATGAAGAGCAAGTA-3′
Reverse primer: XT26 (SEQ ID NO.: 16):
5′-ATTGCGGTACCGCATAAGACCCCTCCA-3′

under the following conditions: 2 min at 95° C., followed by 25 cycles comprising: 45 sec at 55° C., 30 sec at 72° C. and 20 sec at 94° C.

The PCR product was purified, digested by KpnI/EcoRI and cloned into KpnI/EcoRI digested cloning vector p18Xsi to create p18Xsias.

### d) Chimeric XylT Silencing Gene and XylT-RNAi Construct

The assembled sequence (comprising the sense N. benthamiana XylT fragment, intron 2 from A. thaliana XylT gene, and antisense N. benthamiana XylT fragment, totalizing about 840 bp) was removed from p18Xsias by XbaI digestion and cloned into XbaI linearised plant expression vector pGA643 (An et al. (1988) Binary vectors. In S B Gelvin, R A Schilperoort, eds, Plant Molecular Biology Manual, Section A, Chapter 3. Kluwer Academic Publishers, Dordrecht, The Netherlands, pp 1-19), yielding pGAX1 having the nucleotide sequence represented in SEQ ID NO.: 17.

A XylT-RNAi construct (pGAX1) was thus obtained, which comprises:
A chimeric XylT silencing gene comprising:
   a fragment including the promoter region of the Cauliflower Mosaic Virus 35S transcript (Odell et al. (1985) Nature 313: 810) (from nucleotide 12003 to nucleotide 12418 of SEQ ID NO.: 17)
   a fragment including a part of the Nicotiana benthamiana XylT cDNA sequence cloned in sense orientation (304 by long) (from nucleotide 6 to nucleotide 309 of SEQ ID NO.: 17)
   a fragment containing the second intron of the A. thaliana XylT gene (208 bp) (from nucleotide 316 to nucleotide 523 of SEQ ID NO.: 17)
   a fragment including a part of the Nicotiana benthamiana XylT cDNA sequence cloned in antisense orientation (304 by long) (from nucleotide 530 to nucleotide 833 of SEQ ID NO.: 17)
   a fragment including the A. tumefaciens gene 7 terminator as described by (Dhaese et al. (1983) EMBO J. 2: 419-426) (from nucleotide 869 to nucleotide 1090 of SEQ ID NO.: 17)
A chimeric gene encoding a selectable marker comprising:
   a fragment including the promoter region of the nopaline synthase gene of Agrobacterium tumefaciens T-DNA (from nucleotide 9766 to nucleotide 9970 of SEQ ID NO.: 17)
   a fragment including the nptII antibiotic resistance gene (from nucleotide 9971 to nucleotide 10792 of SEQ ID NO.: 17)
   a fragment including the 3′ untranslated region of the nopaline synthase gene of Agrobacterium tumafaciens T-DNA (from nucleotide 11417 to nucleotide 11668 of SEQ ID NO.: 17).
A T-DNA vector backbone comprising:
   the plasmid core comprising the origin of replication from the plasmid pBR322 (Bolivar et al. (1977) Gene 2: 95-113) for replication in Escherichia coli (ORI ColE1)
   a restriction fragment comprising the origin of replication from the Pseudomonas plasmid derivative pTJS75 (Schmidhauser et al. (1985) J. Bact. 164: 446-455) for replication in Agrobacterium tumefaciens and a selectable marker gene conferring resistance to tetracycline resistance (tetR and tetA) for propagation and selection of the plasmid in Escherichia coli and Agrobacterium tumefaciens.
   the right border of the nopaline T-DNA, which is present on an approximately 700 by fragment. This fragment contains the 24-bp conserved sequence that defines one boundary of the transferred DNA (Yadav et al. (1982) Proc. Natl. Acad. Sci. USA 79: 6322-6326) and the overdrive sequence responsible for high efficiency transfer (Peralta et al. (1986) EMBO J. 5: 1137-1142).
   the left border of the nopaline T-DNA, which is present on an approximately 600-bp fragment. This fragment contains both the 24-bp conserved sequence and reduces random termination that is observed when vectors containing no left border are used (Jen and Chilton (1986) Proc. Natl. Acad. Sci. USA 83: 3895-3899).

The resulting XylT-RNAi construct was introduced into Agrobacterium tumefaciens UIA143 comprising helper Ti-plasmid pMP90 (Koncz et al. (1986) Mol. Gen. Genet. 204: 383-396; Farrand et al. (1989) J. Bacteriol. 171: 5314-5321).

### Example 3

### Production and Analysis of XylT-RNAi Nicotiana benthamiana Plants

Nicotiana benthamiana plants were transformed using the Agrobacterium tumefaciens strain described in Example 2 according to the protocol as described in Regner et al. (Plant Cell Reports (1992) 11:22-24).

Primary transformants obtained after leaf disk transformation with the XylT-RNAi construct and selection on appropriate media were tested for genomic insertion of XylT-RNAi sequences using PCR.

Eleven transgenic Nicotiana benthamiana lines, comprising the chimeric gene as described in Example 2 were subsequently subjected to Western blot analyses using xylose specific antibodies to determine the presence/absence of xylose residues. Various staining intensities were obtained for the analyzed plant lines, indicating the presence of different amounts of xylose residues.

One XylT-line (X1) that exhibited very weak staining with corresponding antibodies was grown to maturity.

To monitor changes in the N-glycosylation pattern due to the inactivation of the XylT gene, total endogenous glycoproteins from the XylT-RNAi line (X1) were subjected to total N-glycan analysis by MALDI-TOF mass spectrometry. Absence of xylose and fucose residues on N-glycans, respectively, can be monitored by a reduction of the mass of the respective peaks (132 mass units for xylose, 146 mass units for fucose).

The mass spectrum of total proteins derived from wild-type N. benthamiana plants contained one major peak (1618.4) representing GnGnXF structure. Two minor peaks (1212.0 and 1415.3) were assigned to complex N-glycans structures of MMXF and GnMXF types, respectively. Noteworthy, all three glycoforms contain xylose and fucose (FIG. 1A). The amount of all complex type N-glycans that lacked xylose and fucose residues was assigned below 2%.

Mass spectrometry of total proteins derived from X1 plants (XylT-RNAi plants) differed from that obtained from wild-type N. benthamiana plants in that the major peak (1486.4) was assigned to the complex N-glycan structure GnGnF. Two further peaks were assigned to MMF (1080.0) and GnMF (1283.3). Three minor peaks, just above detection limit, represent complex N-glycans carrying xylose (1212.0: MMXF, 1415.2: GnMXF and 1618.3: GnGnXF) (FIG. 1B). These peaks represent less than 5% of N-glycans indicating the efficient downregulation of XylT in this XylT-RNAi line.

Purified antibodies transiently expressed by Agroinfiltration (Batoko et al. (2000) Plant Cell 12: 2201-2218) in this X1 XylT-RNAi line exhibited a N-glycan profile reflecting the N-glycan composition from their host plant. X1-derived IgGs carried mainly complex N-glycans of GnGnF structures (FIG. 2B). However, minor amounts of complex N-glycans carrying xylose and fucose were still detected on IgGs produced by the XylT-RNAi lines (FIG. 2B and Table 6).

### Example 4

### Isolation of FucT cDNA Sequences from Nicotiana benthamiana

RNA was extracted from leaves of Nicotiana benthamiana using the TRIZOL® Reagent (Invitrogen Life Technologies) according to the manufacturer's protocol and used for cDNA synthesis using SuperScript™ First-strand synthesis System for RT-PCR (Invitrogen Life Technologies) according to the manufacturer's instructions.

Oligonucleotide sequences to be used as degenerated primers in a PCR amplification of FucT cDNA and genomic DNA from Nicotiana benthamiana were designed based on known coding sequences for core α1,3 fucosyltransferases from Arabidopsis thaliana (accession number CAC789979, CAC789980) and Vigna radiata (CAB52254). In this way the following degenerated primers were generated:

Forward: FTAD1 (SEQ ID NO.: 18): 5′-
TGGGC(G/T)GA(A/G)TA(C/T)GATAT(C/T)ATG-3′
Reverse: FTAD2 (SEQ ID NO.: 19): 5′-
GA(A/G)TG(C/T)ACAGC(A/T)GCCATATC-3′

Using the cDNA as template and the above-described pair of primers, a PCR reaction was performed under the following conditions: 5 min at 95° C.; followed by 38 cycles comprising: 15 sec at 94° C. (denaturation), 30 sec at 52° C. (annealing), 2 min at 72° C. (elongation); followed by 4 min at 72° C. (final elongation).

A DNA fragment (partial FucT cDNA) of about 500 basepairs was amplified, cloned into a pCR 2.1-TOPO® vector (Invitrogen) and 2 different clones were obtained yielding to TOPO-FT1 and TOPO-FT2, respectively.

The 5′-end of the cDNA was isolated by carrying out a 5′-RACE PCR using a SMART race KIT (BD Biosciences Clontech, NO. 634914) according to the manufacturer's protocol under the following PCR conditions: 5 cycles comprising 30 sec at 94° C. and 3 min at 72° C.; followed by 5 cycles comprising 30 sec at 94° C., 30 sec at 70° C., and 3 min at 72° C.; followed by 27 cycles comprising 30 sec at 94° C., 30 sec at 68° C., and 3 min at 72° C.; with the following oligonucleotides as primers:

### Forward Primers:

### Universal Primer A MIX Comprising:

Long (SEQ ID NO.: 20):
5′-CTAATACGACTCACTATAGGGCAAGCAGTGGTATCA
ACGCAGAGT-3′
and
Short (SEQ ID NO.: 21): 5′-CTAATACGACTCACTATAGGGC-
3′
Reverse primer: NBFT11 (SEQ ID NO.: 22):
5′-GGATTGACCCAGCTACCAGAGACTGAAAG-3′

The resulting PCR products were subcloned into pGEM-T vector yielding to pGEM-T-Nb-FT-5 end which comprises the 5′-end of FucT cDNA

The 3′-end of the cDNA was isolated by carrying out a 3′-RACE PCR performed using a SMART race KIT (BD Biosciences Clontech, NO. 634914) according to the manufacturer's protocol. This 3′-RACE PCR reaction comprised two successive PCR reactions:

a) a First PCR Reaction Using the Following Oligonucleotides as Primers:

### Forward Primers:

### Universal Primer A MIX Comprising:

Long (SEQ ID NO.: 20):
5′-CTAATACGACTCACTATAGGGCAAGCAGTGGTAT
CAACGCAGAGT-3′
and
Short (SEQ ID NO.: 21): 5′-CTAATACGACTCACTATAGGGC-
3′
Reverse primer: NBFT7 (SEQ ID NO.: 23): 5′-CCTTGGC
AGCGGCTTTCATTTCTAA-3′

under the following PCR conditions: 5 cycles comprising 30 sec at 94° C. and 3 min at 72° C.; followed by 5 cycles comprising 30 sec at 94° C., 30 sec at 68° C., and 3 min at 72° C.; followed by 30 cycles comprising 30 sec at 94° C., 30 sec at 63° C., and 3 min at 72° C.

The resulting PCR product was purified using a NucleoSpin Kit (MN) and used as a template for the successive PCR reaction.

b) a Second PCR Reaction Using the Following Oligonucleotides as Primers:

Forward primer: Nested Universal primer A:
(SEQ ID NO.: 24): 5′-AAGCAGTGGTATCAACGCAGAGT-3′
Reverse Primer: NBFT5 (SEQ ID NO.: 25):
5′-TATACTGCAGTGGTGCTCGCAACTTCCGT-3′

under the following PCR conditions: 30 sec at 94° C., followed by 5 cycles comprising 30 sec at 94° C., 20 sec at 50° C., and 3 min at 72° C.; followed by 25 cycles comprising 20 sec at 94° C., 20 sec at 60° C., and 3 min at 72° C.

The resulting PCR products were subcloned into pGEM-T vector and 2 different clones were obtained which comprise the 3′-end of FucT cDNA, yielding to pGEM-T-Nb-FT-3 end#2 and pGEM-T-Nb-FT-3 end#3, respectively.

The 5′-end, 3′-end, and partial cDNAs obtained above were sequenced and one “complete” FucT-cDNA clone was assembled from sequences of overlapping fragments using the DNASTAR (Seqman/Editseq) software package.

This yielded the “complete” FucT-cDNA nucleotide sequence of 1503 by represented under SEQ ID NO.: 26.

This FucT-cDNA nucleotide sequence encodes a protein of amino acid sequence SEQ ID NO.: 27.

The results of the comparison between the FucT-cDNA nucleotide sequences from Arabidopsis thaliana (accession numbers AJ345084; AJ345085), Lemna minor (DQ789145), Medicago sativa (AY082444; AY082445), Oryza sativa (AK099681), Physcomitrella patens (AJ429145), Vigna radiata (CAB52254) and Zea mays (AY964641), and the FucT cDNA nucleotide sequence isolated from Nicotiana benthamiana (SEQ ID NO.: 26) are presented in Table 3.

TABLE 3

Percentage of identity between the nucleotide sequences of
FucT cDNAs of different plants.

| | | | | | | | | | |
|---|---|---|---|---|---|---|---|---|---|
| | | | | | | Nb FucT | | | |
| Ath | Ath | Lm | Os | Zm | Php | SEQ ID | Ms | Ms | Vr |
| FucT1 | FucT2 | FucT | FucTA | FucT | FucT | NO.: 26 | FucTa | FucTb | FucT |

| | | | | | | | | | |
|---|---|---|---|---|---|---|---|---|---|
| Ath FucT1 | 82 | 64 | 59 | 62 | 55 | 67 | 64 | 65 | 64 |
| Ath FucT2 | | 63 | 60 | 62 | 55 | 68 | 67 | 67 | 67 |
| Lm FucT | | | 66 | 66 | 54 | 64 | 66 | 66 | 65 |
| Os FucTA | | | | 84 | 53 | 64 | 58 | 56 | 56 |
| Zm FucT | | | | | 55 | 64 | 66 | 66 | 66 |
| Php FucT | | | | | | 54 | 55 | 56 | 55 |
| Nb FucT | | | | | | | 74 | 74 | 73 |
| SEQ ID | | | | | | | | | |
| NO.: 26 | | | | | | | | | |
| Ms FucTa | | | | | | | | 98 | 76 |
| Ms FucTb | | | | | | | | | 73 |
| Vr FucT | | | | | | | | | |

Abbreviations:
Ath: Arabidopsis thaliana,
Nb: Nicotiana benthamiana,
Ms: Medicago sativa,
Lm: Lemna minor,
Os: Oryza sativa,
Zm: Zea mays,
Php: Physcomitrella patens,
Vr: Vigna radiata.

The results of the comparison between the amino acid sequence of the putative FucT protein from Arabidopsis thaliana (accession number CAC78979, CAC78980), Lemna minor (ABG89268.), Medicago sativa (AAL99370; AAL99371), Oryza sativa (BAD09365), Physomitrella patens (Q8L5D1), and Zea mays (Q0VH31), and from Nicotiana benthamiana (SEQ ID NO.: 27) are presented in Table 4.

TABLE 4

Percentage of identity between the amino acid sequences
of FucT protein of different plants.

| | | | | | | | |
|---|---|---|---|---|---|---|---|
| | | | | | | | Nb FucT |
| | | | | | | | SEQ ID NO.: |
| Ath FucT2 | Lm FucT | Os FucT | Zm FucT | Php FucT | Ms FucTa | Ms FucTb | 27 |

| | | | | | | | | |
|---|---|---|---|---|---|---|---|---|
| Ath FucT1 | 78 | 59 | 60 | 60 | 49 | 66 | 66 | 64 |
| Ath FucT2 | | 59 | 60 | 60 | 48 | 66 | 66 | 64 |
| Lm FucT | | | 62 | 64 | 49 | 64 | 64 | 61 |
| Os FucT | | | | 85 | 47 | 62 | 62 | 59 |
| Zm FucT | | | | | 48 | 62 | 62 | 59 |
| Php FucT | | | | | | 50 | 50 | 48 |
| Ms FucTa | | | | | | | 99 | 69 |
| Ms FucTb | | | | | | | | 69 |

Abbreviations:
Ath: Arabidopsis thaliana,
Nb: Nicotiana benthamiana,
Ms: Medicago sativa,
Lm: Lemna minor,
Os: Oryza sativa,
Zm: Zea mays,
Php: Physcomitrella patens.

### Example 5

### Construction of a T-DNA Vector Containing a Nicotiana benthamiana FucT Silencing Gene (FucT-RNAi Construct)

DNA fragments amplified from Nicotiana benthamiana FucT sequences described in Example 4 were used to construct T-DNA vectors comprising a chimeric gene which upon transcription yields an RNA molecule comprising a sense and antisense FucT DNA sequence from the amplified DNA fragment, and which could basepair to form a double stranded RNA molecule. Such a chimeric gene can be used to reduce the expression of a FucT gene in Nicotiana, particularly in Nicotiana benthamiana.

a) Cloning of the Intron 2 from the A. thaliana XylT Gene

First, a XylT DNA fragment from the A. thaliana XylT gene (Accession number At5g55500) was amplified by PCR using the genomic DNA from A. thaliana ecotype Wassilewskija WS-3 as template as described in Example 2.

The PCR product was purified, digested by BamHI and KpnI and ligated into BamHI/KpnI digested cloning vector puc18, leading to vector p18I2.

### b) Cloning of the Sense FucT Sequence

Oligonucleotide sequences to be used as non-degenerated primers in a PCR amplification of a FucT gene sequence from Nicotiana benthamiana were designed based on the cDNA sequence from Nicotiana benthamiana isolated in Example 4.

The sense FucT fragment was produced by PCR using the vector Topo-FT-1 comprising a cDNA fragment amplified from Nicotiana benthamiana leaf mRNA as described in Example 4 (NbFT-1) as template and the following primers:

Forward primer: NbFT1 (SEQ ID NO.: 28):
5′-TTATGGTACCGGATCCTTGGCAGCGGCTTTCATTT-3′
Reverse primer: NbFT2 (SEQ ID NO.: 29):
5′-AATTGGTACCGGATCCATCAGATGGGCCCTCAAACT-3′

under the following conditions: 2 min at 95° C., followed by 25 cycles comprising: 45 sec at 55° C., 30 sec at 72° C. and 20 sec at 94° C.

The PCR product was purified, BamHI digested and cloned into BamHI digested cloning vector p18I2 to create puc18Fsi.

### c) Cloning of the Antisense FucT Sequence

The antisense FucT fragment was produced by PCR also using the vector Topo-FT-1 as template but with the following primers:

Forward primer: NbFT2 (SEQ ID NO.: 29):
5′-AATTGGTACCGGATCCATCAGATGGGCCCTCAAACT-3′
Reverse primer: NbFT4 (SEQ ID NO.: 30):
5′-TTATGGTACCTCTAGATTGGCAGCGGCTTTCATTT-3′

under the following conditions: 2 min at 95° C., followed by 25 cycles comprising: 45 sec at 55° C., 30 sec at 72° C. and 20 sec at 94° C.

The PCR product was purified, digested by KpnI and cloned into KpnI digested cloning vector p18Fsi to create puc18Fsias.

### d) Chimeric FucT Silencing Gene and FucT-RNAi Construct

The assembled sequence (comprising the sense N. benthamiana FucT fragment, intron 2 from A. thaliana XylT gene, and antisense N. benthamiana FucT fragment, totalizing about 1070 bp) was removed from puc18Fsias by XbaI digestion and cloned into XbaI linearised plant expression vector pGA643 (An et al. (1988) Binary vectors. In S B Gelvin, R A Schilperoort, eds, Plant Molecular Biology Manual, Section A, Chapter 3. Kluwer Academic Publishers, Dordrecht, The Netherlands, pp 1-19) yielding pGAX3 having the nucleotide sequence represented under SEQ ID NO.: 31.

A FucT-RNAi construct pGAX3 was thus obtained, which comprises:
A chimeric FucT silencing gene comprising:
   a fragment including the promoter region of the Cauliflower Mosaic Virus 35S transcript (Odell et al. (1985) Nature 313: 810) (from nucleotide 11169 to nucleotide 11584 of SEQ ID NO.: 31)
   a fragment including a part of the Nicotiana benthamiana FucT cDNA sequence cloned in sense orientation (426 by long) (from nucleotide 11602 to nucleotide 12027 of SEQ ID NO.: 31)
   a fragment containing the second intron of the A. thaliana XylT gene (218 bp) (from nucleotide 12028 to nucleotide 12245 of SEQ ID NO.: 31)
   a fragment including a part of the Nicotiana benthamiana FucT cDNA sequence cloned in antisense orientation (420 by long) (from nucleotide 12248 to nucleotide 12667 of SEQ ID NO.: 31)
   a fragment including the A. tumefaciens gene 7 terminator as described by (Dhaese et al. (1983) EMBO J. 2: 419-426) (from nucleotide 35 to nucleotide 246 of SEQ ID NO.: 31)
A chimeric gene encoding a selectable marker comprising:
   A fragment including the promoter region of the nopaline synthase gene of Agrobacterium tumefaciens T-DNA (from nucleotide 8932 to nucleotide 9136 of SEQ ID NO.: 31)
   A fragment including the nptII antibiotic resistance gene (from nucleotide 9137 to nucleotide 9958 of SEQ ID NO.: 31)
   A fragment including the 3′ untranslated region of the nopaline synthase gene of Agrobacterium tumafaciens T-DNA (from nucleotide 10583 to nucleotide 10834 of SEQ ID NO.: 31).
A T-DNA vector backbone comprising:
   the plasmid core comprising the origin of replication from the plasmid pBR322 (Bolivar et al. (1977) Gene 2: 95-113) for replication in Escherichia coli (ORI ColE1)
   a restriction fragment comprising the origin of replication from the Pseudomonas plasmid derivative pTJS75 (Schmidhauser et al. (1985) J. Bact. 164: 446-455) for replication in Agrobacterium tumefaciens and a selectable marker gene conferring resistance to tetracycline resistance (tetR and tetA) for propagation and selection of the plasmid in Escherichia coli and Agrobacterium tumefaciens.
   the right border of the nopaline T-DNA, which is present on an approximately 700 by fragment. This fragment contains the 24-bp conserved sequence that defines one boundary of the transferred DNA (Yadav et al. (1982) Proc. Natl. Acad. Sci. USA 79: 6322-6326) and the overdrive sequence responsible for high efficiency transfer (Peralta et al. (1986) EMBO J. 5: 1137-1142).
   the left border of the nopaline T-DNA, which is present on an approximately 600-bp fragment. This fragment contains both the 24-bp conserved sequence and reduces random termination that is observed when vectors containing no left border are used (Jen and Chilton (1986) Proc. Natl. Acad. Sci. USA 83: 3895-3899).

The resulting FucT-RNAi construct was introduced into Agrobacterium tumefaciens UIA143 comprising helper Ti-plasmid pMP90 (Koncz et al. (1986) Mol. Gen. Genet. 204: 383-396; Farrand et al. (1989) J. Bacteriol. 171: 5314-5321).

### Example 6

### Production and Analysis of FucT-RNAi Nicotiana benthamiana Plants

Nicotiana benthamiana plants were transformed using the Agrobacterium tumefaciens strain described in Example 5 according to the protocol as described in Regner et al. (Plant Cell Reports (1992) 11: 22-24).

Primary transformants obtained after leaf disk transformation with the FucT-RNAi construct and selection on appropriate media were tested for genomic insertion of FucT-RNAi sequences using PCR.

Nine transgenic Nicotiana tabacum lines, comprising the chimeric gene as described in Example 5 were subsequently subjected to Western blot analyses using fucose specific antibodies to determine the presence/absence of fucose residues. Various staining intensities were obtained for the analyzed plant lines, indicating the presence of different amounts of fucose residues.

One FucT-line (F3) that exhibited very weak staining with corresponding antibodies was grown to maturity.

To monitor changes in the N-glycosylation pattern due to the inactivation of the FucT gene, soluble endogenous proteins from the FucT-RNAi line (F3) were subjected to total N-glycan analysis by MALDI-TOF mass spectrometry. Absence of xylose and fucose residues on N-glycans, respectively, can be monitored by a reduction of the mass of the respective peaks (132 mass units for xylose, 146 mass units for fucose).

The mass spectrum of total soluble endogenous proteins derived from wild-type N. benthamiana plants contained one major peak (1618,4) representing GnGnXF structure. Two minor peaks (1212.0 and 1415.3) were assigned to complex type N-glycans of MMXF and GnMXF type, respectively (FIG. 1A). Noteworthy, all three glycoforms contain xylose and fucose. Therefore, the amount of all complex type N-glycans that lacked xylose and fucose residues was assigned below 2%.

The result of mass spectrometry analysis of total soluble endogenous proteins derived from F3 plants (FucT-RNAi plants) differed from that obtained from wild-type N. benthamiana plants in that three major peaks (1066, 1269, and 1472.2) were assigned to the complex N-glycan structures MMX, GnMX, and GnGnX, respectively. However, by contrast to X1, two additional peaks (1212 and 1618.1) that were assigned to the complex N-glycan structures MMXF and GnGnXF were clearly detectable indicating that the reduction of fucose in F3 was not as efficient as was the reduction of xylose in X1 (FIG. 1C).

Purified antibodies transiently expressed by Agroinfiltration in this F3 FucT-RNAi line exhibit a N-glycan profile reflecting the N-glycan composition from their host plant. F3-derived IgGs carry mainly complex N-glycans of GnGnX structures (FIG. 2C). However, minor amounts of complex N-glycans carrying xylose and fucose were still detected on IgGs produced by the FucT-RNAi line.

### Example 7

### Preparation of XylT-FucT-RNAi Nicotiana benthamiana Plants Capable of Expressing IgG Antibodies without Detectable β-1,2-Xylose Residues and α-1,3 Fucose Residues on N-Glycans Bound to Said Antibodies

X1 and F3 lines, produced in Examples 3 and 6, respectively, were crossed and the progeny thereof was screened by genomic PCR (gPCR) for the presence of both XylT and FucT RNAi sequences.

Positive gPCR plants were subjected to Western blotting using anti-horseradish peroxidase antibodies which recognise β1,2-xylose- and core α-1,3-fucose-containing epitopes (Wilson et al. (1998) Glycobiology 8: 651-661). One plant that exhibited no signal (C100) was selected for antibody expression.

C100 plant cells were transiently transformed by infiltration of leaves with an Agrobacterium tumefaciens strain harboring a plasmid comprising genes coding for the light and heavy chains of a human IgG.

### Example 8

### Analysis of XylT-FucT-RNAi Nicotiana benthamiana Plants Expressing IgG Antibodies without Detectable β-1,2-Xylose Residues and α-1,3 Fucose Residues on N-Glycans Bound to Said Antibodies

Although minor peaks that represent complex N-glycans carrying xylose and fucose were detected when total soluble endogenous proteins were analysed (FIG. 1D, Table 5), no fractions carrying β-1,2 xylose and/or α-1,3 fucose complex N-glycans were detected on IgG transiently expressed in these plant cells by Agroinfiltration (FIG. 2D).

TABLE 5
Mass spectrometry analysis of N-glycans of total endogenous proteins
from wild-type (control) N. benthamiana plant, X1 (XylT-RNAi)
N. benthamiana plant, F3 (FucT-RNAi) N. benthamiana
plant, and C100 (XylT-FucT-RNAi) N. benthamiana plant.
Values indicate the relative abundance of a specific glycoform (%).

| N-glycans | | | | |
|---|---|---|---|---|
| containing | wild-type | X1 | F3 | C100 |
| α-1,3 fucose | 67 | 51 | 22 | 20 |
| β-1,2 xylose | 81 | <3 | 74 | <3 |

TABLE 6
Mass spectrometry analysis of N-glycans of purified IgG heavy chain
from wild-type (control) N. benthamiana plant, X1 (XylT-RNAi)
N. benthamiana plant, F3 (FucT-RNAi) N. benthamiana
plant, and C100 (XylT-FucT-RNAi) N. benthamiana plant.
Values indicate the relative abundance of a specific glycoform (%).

| N-glycan | wild-type | X1 | F3 | C100 |
|---|---|---|---|---|
| GnGn | 1.9 | 20.8 | 5.8 | 72.6 |
| GnGnF | 2.1 | 39.5 | 1.2 | <1 |
| GnGnX | 5.5 | 1.3 | 41.8 | <1 |
| GnGnXF | 67.6 | 10.4 | 19.4 | <1 |

Similar results were obtained when progeny of C100, which represent a mixture of heterozygous offspring, were analysed.

In summary, complex N-glycans decorated with still detectable amounts of xylose and fucose were detected when total proteins were analysed in C100 plant cells. In contrast, IgGs produced in these plants carry complex N-glycan structures (mostly GnGn) without detectable xylose and/or fucose residues.

Additionally, plant derived IgGs were subjected to immunoblotting using antibodies that recognize plant specific glycan epitopes (anti-HRP antibody). As expected a single band with a molecular mass of about 55 kDa, representing the size of the heavy chain, was detected on the IgGs produced by wild-type N. benthamiana, even with an amount as low as 10 ng IgG. In contrast, no signal was detected in C100-derived IgGs, even with high amounts such as 400 ng IgG, indicating the absence of immunogenic glycan epitopes.

An additional benefit of producing monoclonal antibodies with the method described in the present invention is that the produced antibodies exhibit a widely homogenous N-glycan profile. Indeed, over 70% of complex N-glycans were homogenous GnGn structure (Table 6). This constitutes an advantage over CHO produced antibodies, where a variety of glycoforms are present.

Antibodies derived from wild-type N. benthamiana and XylT-FucT-RNAi lines were undistinguishable from CHO derived IgG in respect to electrophoretic properties and assembly (data not shown).

XylT-FucT-RNAi lines are viable and revealed no obvious morphological phenotype under standard growth condition and during the Agroinfiltration process. Furthermore, IgG expression levels were comparable between RNAi lines and wild-type N. benthamiana indicating the suitability of these transformed plants for the production of antibodies lacking immunogenic N-glycan residues.

### Example 9

### Protocol Used for Analyzing Recombinant Antibodies in the Above Examples

### a) Purification of Recombinant IgG

Leaves were frozen in liquid nitrogen and ground in a mixer mill (Retsch MM2000). The powder (200 mg) was dissolved in 400 μl of 1×PBS (pH 6). After centrifugation for 30 min at 16.000×g, the supernatant (SN1) was incubated at 4° C. for 90 min with 15 μl rProteinA Sepharose Fast Flow (GE Healthcare) using an orbital shaker. The incubated slurry was transferred into Micro Bio-Spin chromatography column (Biorad) and washed 3 times with 250 μl 1×PBS. Elution was performed with 15 μl 0.1 M glycine-HCl buffer (pH 3.0).

### b) Immunoblot Analysis

SN1 and Purified IgG1 (100 ng) samples were subjected to SDS-PAGE (12.5% polyacrylamide) under reducing conditions. The separated proteins were blotted to Hybond-ECL membranes (GE Healthcare) and detected either with a goat anti-human IgG (C_{H}+C_{L} specific) antibody conjugated to peroxidase (Promega) or goat anti-human IgG (γ-chain specific) antibody conjugated to peroxidase (Sigma, A8775) both 1:5000 diluted in 1×PBS (pH 7.4) containing 1% (w/v) BSA. For detection of N-linked glycans with β1,2-xylose and core α1,3-fucose rabbit anti-horseradish-peroxidase antibody (anti-HRP) was used as described (Strasser et al. (2004) FEBS Lett. 561:132-136). Detection of bound antibodies was performed using SuperSignal West Pico Chemiluminescent substrate (Pierce).

### c) N-Glycan Analysis by Liquid-Chromatography-ElectroSpray Ionization-Mass Spectrometry

Purified IgG1 (0.5 μg) was separated by SDS-PAGE (12.5% polyacrylamide) analysis under reducing conditions and polypeptides were detected by Coomassie Blue staining. The heavy chain was excised from the gel, destained, carbamidomethylated and in-gel trypsin-digested as described (Kolarich et al. (2000) Anal. Biochem. 285: 64-75). Tryptic peptides were dried in a Speed Vac concentrator and reconstituted with water containing 0.1% (v/v) formic acid. Mass spectrometric analysis was performed on a Q-TOF Ultima Global (Waters Micromass) equipped with a standard electro-spray unit, a Cap-LC system (Waters Micromass) and a 10-port solvent switch module (Rheodyne). Samples were at first captured by an Aquasil C18 pre-column (30×0.32 mm, Thermo Electron) using water as the solvent. The analytical column was held at 5% acetonitrile before solvent switching and then a linear gradient from 5 to 50% acetonitrile was applied at a flow rate of 2 μl/min. All eluents contained 0.1% formic acid. The mass spectrometer had been previously tuned with [Glu1]-fibrino-peptide B to give the highest possible sensitivity and a resolution of ca. 10.000 (FWHM). Mass tuning of the TOF analyser was performed in the tandem MS mode using again [Glu1]-fibrinopeptide B. Samples were analysed in the MS mode. Because no switching between MS and tandem MS mode was performed, no loss of signal, especially for the analysis of the glycopeptides, occurred. Data analysis was performed with MassLynx 4.0 SP4 Software (Waters Micromass).

The Mass spectrometry data of tryptic peptides were analysed against the in silico generated tryptic digestion of the IgG C_{H} amino acid sequence, employing the program “PeptideMass” (http://www.expasy.org/tools/peptide-mass.html). Based on the tryptic peptide data set, the tryptic glycopeptide data-sets (“glycopeptide 1” and “glycopeptide 2”, representing the “perfectly” cleaved tryptic glycopeptide “EEQYNSTYR”, and the tryptic glycopeptide bearing one missed cleavage site “TKPREEQYNSTYR”, respectively) were generated by the addition of the respective glycan masses to the tryptic peptide masses of glycopeptide 1 and glycopeptide 2.

Total protein N-glycans from N. benthamiana leaves were prepared and analysed as reported in Wilson et al. (2001) (Glycobiology 11: 261-274).

1. A method to produce a plant cell or plant having a low level of β-1,2-xylose residues and core α-1,3-fucose residues on protein-bound N-glycans comprising crossing a first plant of having a low level of β-1,2-xylose residues on protein-bound N-glycans with a second plant having low level of core α-1,3 fucose residues on protein-bound N-glycans, and
optionally, identifying from the progeny obtained from said crossing a plant which has a low level of β-1,2-xylose and core α-1,3-fucose residues on protein-bound N-glycans;
wherein at least one gene encoding a β-1,2-xylosyltransferase in said first plant and at least one gene encoding an α-1,3-fucosyltransferase in said second plant have not been disrupted, deleted, or inactivated by mutagenesis such as substitution, deletion or insertion.

2. The method of claim 1, wherein the first plant and the second plant are Nicotiana plants.

3. The method of claim 2, wherein the first plant and the second plant are from the same Nicotiana species or cultivar.

4. The method of claim 3, wherein the Nicotiana species is Nicotiana benthamiana.

5. The method of claim 1, wherein said low level of β-1,2-xylose residues on protein-bound N-glycans in the first plant comprises transcriptional or post-transcriptional silencing of the expression of the endogenous β-1,2 xylosyltransferase encoding gene(s); and said low level of α-1,3-fucose residues on protein-bound N-glycans in the second plant comprises transcriptional or post-transcriptional silencing of the expression of the endogenous α-1,3-fucosyltransferase encoding gene(s).

6. The method of claim 5, wherein:
said first plant is produced by the method comprising the steps of:
(a) transforming a plant cell with a first chimeric gene comprising the following operably linked DNA fragments:
(i) a plant expressible promoter;
(ii) a DNA region which, when transcribed, yields an RNA molecule capable of forming a double stranded RNA region at least between:
(I) an RNA region transcribed from a first sense DNA region comprising a nucleotide sequence of at least 18 out of 20-21 consecutive nucleotides comprising a nucleotide sequence encoding a XyIT protein, or the complement thereof, said nucleotide sequence obtainable from the same species or cultivar as the plant cells into which the first chimeric gene is to be introduced, or selected from a nucleotide sequence of a XyIT gene or a XyIT cDNA, or the complement thereof, said nucleotide sequence obtainable from the same species or cultivar as the plant cells into which the first chimeric gene is to be introduced;
(II) an RNA region transcribed from a second antisense DNA region comprising a nucleotide sequence of at least 18 consecutive nucleotides which have at least 95% sequence identity to the complement of said first sense DNA region; and
(iii) a DNA region comprising a transcription termination and polyadenylation signal functional in plants;
(b) optionally, identifying a transgenic plant cell which has a lower level of β-1,2-xylose residues on protein-bound N-glycans than an untransformed plant cell;
(c) regenerating one or more transgenic plant cells from step (a) or (b) to obtain transgenic plants;
(d) optionally, identifying a transgenic plant which has a lower level of β-1,2-xylose residues on protein-bound N-glycans than an untransformed plant; and
said second plant is produced by the method comprising the steps of:
(a) transforming a plant cell with a second chimeric gene comprising the following operably linked DNA fragments:
(i) a plant expressible promoter;
(ii) a DNA region which, when transcribed, yields an RNA molecule capable of forming a double stranded RNA region at least between
(I) an RNA region transcribed from a third sense DNA region comprising a nucleotide sequence of at least 18 out of 20-21 consecutive nucleotides comprising a nucleotide sequence encoding a FucT protein, or the complement thereof, said nucleotide sequence obtainable from the same species or cultivar as the plant cells into which the second chimeric gene is to be introduced, or selected from a nucleotide sequence of a FucT gene or a FucT cDNA, or the complement thereof, said nucleotide sequence obtainable from the same species or cultivar as the plant cells into which the second chimeric gene is to be introduced;
(II) an RNA region transcribed from a fourth antisense DNA region comprising a nucleotide sequence of at least 18 consecutive nucleotides which have at least 95% sequence identity to the complement of said third sense DNA region; and
(iii) a DNA region comprising a transcription termination and polyadenylation signal functional in plants;
(b) optionally, identifying a transgenic plant cell which has a lower level of core α-1,3-fucose residues on protein-bound N-glycans than an untransformed plant cell;
(c) regenerating one or more transgenic plant cells from step a) or b) (a) or (b) to obtain transgenic plants; and
(d) optionally, identifying a transgenic plant which has a lower level of core α-1,3-fucose residues on protein-bound N-glycans than an untransformed plant.

7. The method according to claim 5, wherein:
said first plant is produced by the method comprising the steps of:
(a) transforming a plant cell with a first chimeric gene to generate transgenic plant cells, said first chimeric gene comprising the following operably linked DNA fragments:
(i) a plant expressible promoter;
(ii) a DNA region comprising a nucleotide sequence of at least 18 out of 20-21 consecutive nucleotides selected from a nucleotide sequence encoding a XyIT protein, or the complement thereof, said nucleotide sequence obtainable from the same species or cultivar as the plant cells into which said first chimeric gene is to be introduced, or selected from a nucleotide sequence of a XyIT gene or a XyIT cDNA, or the complement thereof, said nucleotide sequence obtainable from the same species or cultivar as the plant cells into which said chimeric gene is to be introduced, in antisense or sense orientation;
(iii) a DNA region comprising a transcription termination and polyadenylation signal functional in plants;
(b) optionally, identifying a transgenic plant cell which has a lower level of β-1,2-xylose residues on protein-bound N-glycans than an untransformed plant cell;
(c) regenerating one or more transgenic plant cells from step (a) or (b) to obtain transgenic plants;
(d) optionally, identifying a transgenic plant which has a lower level of β-1,2-xylose residues on protein-bound N-glycans than an untransformed plant; and
said second plant is produced by the method comprising the steps of:
(a) transforming a plant cell with a second chimeric gene to generate transgenic plant cells, said second chimeric gene comprising the following operably linked DNA fragments:
(i) a plant expressible promoter;
(ii) a DNA region comprising a nucleotide sequence of at least 18 out of 20-21 consecutive nucleotides comprising a nucleotide sequence encoding a FucT protein, or the complement thereof, said nucleotide sequence obtainable from the same species or cultivar as the plant cells into which said second chimeric gene is to be introduced, or selected from a nucleotide sequence of a FucT gene or a FucT cDNA, or the complement thereof, said nucleotide sequence obtainable from the same species or cultivar as the plant cells into which said second chimeric gene is to be introduced, in the antisense or sense orientation; and
(iii) a DNA region comprising a transcription termination and polyadenylation signal functional in plants;
(b) optionally, identifying a transgenic plant cell which has a lower level of core α-1,3-fucose residues on protein-bound N-glycans than an untransformed plant cell;
(c) regenerating one or more transgenic plant cells from step (a) or (b) to obtain transgenic plants; and
(d) optionally, identifying a transgenic plant which has a lower level of core α-1,3-fucose residues on protein-bound N-glycans than an untransformed plant.

8. The method according to claim 5, wherein:
said first plant is produced by the method comprising the steps of:
(a) providing one or more first double stranded RNA molecules to plant cells or to a plant, wherein the first double stranded RNA molecule(s) comprise two RNA strands, one RNA strand consisting essentially of an RNA nucleotide sequence of at least 18 out of 20-21 consecutive nucleotides selected from a nucleotide sequence encoding a XyIT protein, or the complement thereof, said nucleotide sequence obtainable from the same species or cultivar as the cells of the plant into which the first double stranded RNA molecule(s) is to be introduced, or selected from the nucleotide sequence of a XyIT gene or a XyIT cDNA, or the complement thereof, said nucleotide sequence obtainable from the same species or cultivar as the plant cells into which said first double stranded RNA molecule(s) is to be introduced;
(b) identifying a transformed plant cell comprising said first double stranded RNA molecule(s) which has a lower level of β-1,2-xylose residues on protein-bound N-glycans than an untransformed plant cell;
(c) optionally, regenerating one or more transformed plant cells from step (a) or (b) to obtain transformed plants;
(d) identifying, from the transformed plants obtained in step (a) or (c), a transformed plant which has a lower level of β-1,2-xylose residues on protein-bound N-glycans than an untransformed plant; and
said second plant is produced by the method comprising the steps of:
(a) providing one or more second double stranded RNA molecules to plant cells or to a plant, wherein the second double stranded RNA molecules comprise two RNA strands, one RNA strand consisting essentially of an RNA nucleotide sequence of at least 18 out of 20-21 consecutive nucleotides selected from a nucleotide sequence encoding a FucT protein, or the complement thereof, said nucleotide sequence obtainable from the same species or cultivar as the cells of the plant into which the second double stranded RNA molecule(s) is to be introduced, or selected from the nucleotide sequence of a FucT gene or a FucT cDNA, or the complement thereof, said nucleotide sequence obtainable from the same species or cultivar as the plant cells into which said second double stranded RNA molecule(s) is to be introduced;
(b) optionally, identifying a transformed plant cell comprising said second double stranded RNA molecule(s) which has a lower level of core α-1,3-fucose residues on protein-bound N-glycans than an untransformed plant cell;
(c) optionally, regenerating one or more transformed plant cells from step (a) or (b) to obtain transformed plants;
(d) identifying, from the transformed plants obtained in step (a) or (c), a transformed plant which has a lower level of α-1,3-fucose residues on protein-bound N-glycans than an untransformed plant.

9. The method according to claim 8, wherein:
the first double stranded RNA of the first plant is provided to said plant cells by integrating a first chimeric gene into the genome of said plant cells to generate transgenic plant cells, and said transgenic plant cells are regenerated to obtain transgenic plants, said first chimeric gene comprising the following operably linked DNA fragments:
(a) a plant expressible promoter;
(b) a DNA region which, when transcribed, yields an RNA molecule capable of forming a double stranded RNA region at least between
(i) an RNA region transcribed from a first sense DNA region comprising a nucleotide sequence of at least 18 out of 20-21 consecutive nucleotides selected from a nucleotide sequence encoding a XyIT protein, or the complement thereof, said nucleotide sequence obtainable from the same species or cultivar as the plant cells in the genome of which the first chimeric gene is to be integrated, or selected from a nucleotide sequence of a XyIT gene or a XyIT cDNA, or the complement thereof, said nucleotide sequence obtainable from the same species or cultivar as the plant cells in the genome of which said first chimeric gene is to be integrated; and
(ii) an RNA region transcribed from a second antisense DNA region comprising a nucleotide sequence of at least 18 consecutive nucleotides which have at least 95% sequence identity to the complement of said first sense DNA region; and
(c) a DNA region comprising a transcription termination and polyadenylation signal functional in plants; and
the second double stranded RNA of the second plant is provided to said plant cells by integrating a second chimeric gene into the genome of said plant cells to generate transgenic plant cells, and said transgenic plant cells are regenerated to obtain transgenic plants, said second chimeric gene comprising the following operably linked DNA fragments:
(a) a plant expressible promoter;
(b) a DNA region which, when transcribed, yields an RNA molecule capable of forming a double stranded RNA region at least between
(i) an RNA region transcribed from a third sense DNA region comprising a nucleotide sequence of at least 18 out of 20-21 consecutive nucleotides selected from a nucleotide sequence encoding a FucT protein, or the complement thereof, said nucleotide sequence obtainable from the same species or cultivar as the plant cells in the genome of which said second chimeric gene is to be integrated, or selected from a nucleotide sequence of a FucT gene or a FucT cDNA, or the complement thereof, said nucleotide sequence obtainable from the same species or cultivar as the plant cells in the genome of which said second chimeric gene is to be integrated; and
(ii) an RNA region transcribed from a fourth antisense DNA region comprising a nucleotide sequence of at least 18 consecutive nucleotides which have at least 95% sequence identity to the complement of said third sense DNA region; and iii) a DNA region comprising a transcription termination and polyadenylation signal functional in plants.

10. The method of claim 8, wherein:
the first double stranded RNA of the first plant is provided to said plant cells by integrating a chimeric gene into the genome of said plant cells to generate transgenic plant cells, and said transgenic plant cells are regenerated to obtain transgenic plants, said chimeric gene comprising the following operably linked DNA fragments:
(a) a plant expressible promoter;
(b) a DNA region comprising at least 18 out of 20-21 consecutive nucleotides selected from a nucleotide sequence encoding a XyIT protein, or the complement thereof, said nucleotide sequence obtainable from the same species or cultivar as the plant cells in the genome of which said chimeric gene is to be integrated, or selected from the nucleotide sequence of a XyIT gene or a XyIT cDNA, or the complement thereof, said nucleotide sequence obtainable from the same species or cultivar as the plant cells in the genome of which said chimeric gene is to be integrated, in antisense or sense orientation;
(c) a DNA region comprising a transcription termination and polyadenylation signal functional in plants; and
the second double stranded RNA of the second plant is provided to said plant cells by integrating a chimeric gene into the genome of said plant cells to generate transgenic plant cells, and said transgenic plant cells are regenerated to obtain transgenic plants, said chimeric gene comprising the following operably linked DNA fragments:
(a) a plant expressible promoter;
(b) a DNA region comprising at least 18 out of 20-21 consecutive nucleotides selected from a nucleotide sequence encoding a FucT protein, or the complement thereof, said nucleotide sequence obtainable from the same species or cultivar as the plant cells in the genome of which said chimeric gene is to be integrated, or selected from the nucleotide sequence of a FucT gene or a FucT cDNA, or the complement thereof, said nucleotide sequence obtainable from the same species or cultivar as the plant cells in the genome of which said chimeric gene is to be integrated, in antisense or sense orientation; and
(c) a DNA region comprising a transcription termination and polyadenylation signal functional in plants.

11. The method of claim 6, wherein said XyIT protein comprises a nucleotide sequence encoding the amino acid sequence of SEQ ID NO.: 10.

12. The method of claim 6, wherein said nucleotide sequence of a XyIT gene or a XyIT cDNA comprises the sequence of SEQ ID NO.: 9.

13. The method of claim 6, wherein said DNA regions comprise at least 50 of said consecutive nucleotides.

14. The method of claim 6, wherein said DNA regions comprise at least 100, or at least 150, or at least 200 of said consecutive nucleotides.

15. The method of claim 1 comprising
deleting, disrupting, or replacing the endogenous XyIT gene(s) and integrating an exogenous XyIT allele correlated with a low level of β-1,2-xylose residues on protein-bound N-glycans; and further comprising
deleting, disrupting, or replacing the endogenous FucT gene(s) and integrating an exogenous FucT allele correlated with a low level of α-1,3-fucose residues on protein-bound N-glycans.

16. The method of claim 1 further comprising the step of crossing said plant having a low level of β-1,2-xylose residues and α-1,3-fucose residues on protein-bound N-glycans to a second plant to obtain progeny plants having a low level of β-1,2-xylose residues and α-1,3-fucose residues on protein-bound N-glycans.

17-19. (canceled)

20. A method of producing a foreign glycoprotein of interest having a low level of, or no detectable, β-1,2-xylose and α-1,3-fucose residues on N-glycans bound to said foreign glycoprotein, comprising:
(a) producing a plant cell or plant having a low level of β-1,2-xylose residues and core α-1,3-fucose residues on protein-bound N-glycans according to claim 1;
(b) providing to a plant cell or plant obtained in step (a) a chimeric gene comprising the following operably linked DNA fragments: a plant expressible promoter, a DNA region encoding the glycoprotein of interest, and a DNA region comprising a transcription termination and polyadenylation signal functional in plants;
(c) optionally, identifying a transformed plant or plant cell expressing the glycoprotein of interest;
(d) cultivating the transformed plant or plant cell obtained in step (c);
(e) optionally, extracting and purifying the foreign glycoprotein of interest from the total plant proteins.

21. The method of claim 20, wherein the plant cell or plant is from a Nicotiana species or cultivar.

22. A method to identify a Nicotiana FucT DNA fragment comprising the steps of:
(a) providing genomic DNA or cDNA obtainable from a Nicotiana species or cultivar;
(b) selecting any one of the following probes or primers:
(i) a DNA fragment comprising a nucleotide sequence encoding the amino acid sequence of SEQ ID NO.: 27;
(ii) a DNA fragment comprising the nucleotide sequence of SEQ ID NO.: 26;
(iii) a DNA fragment or oligonucleotide comprising a nucleotide sequence consisting of between 20 to 200 consecutive nucleotides of a nucleotide sequence encoding the amino acid sequence of SEQ ID NO.: 27;
(iv) a DNA fragment or oligonucleotide comprising a nucleotide sequence consisting of between 20 to 1503 consecutive nucleotides of a nucleotide sequence encoding the amino acid sequence of SEQ ID NO.: 27;
(v) a DNA fragment or oligonucleotide comprising a nucleotide sequence consisting of between 20 to 200 consecutive nucleotides of a nucleotide sequence of SEQ ID NO.: 26;
(vi) a DNA fragment or oligonucleotide comprising a nucleotide sequence consisting of between 20 to 1503 consecutive nucleotides of a nucleotide sequence of SEQ ID No.: 26;
(vii) an oligonucleotide having a nucleotide sequence comprising between 20 to 200 consecutive nucleotides of a nucleotide sequence encoding the amino acid sequence of SEQ ID NO.: 27;
(viii) an oligonucleotide comprising a nucleotide sequence comprising between 20 to 200 consecutive nucleotides of the nucleotide sequence of SEQ ID NO.: 26; or
(ix) an oligonucleotide comprising the nucleotide sequence of any one of SEQ ID NO.: 28 and SEQ ID NO.: 29;
(c) identifying a FucT DNA fragment from said Nicotiana species or cultivar by performing a PCR reaction using said genomic DNA, cDNA, and primers, or by performing hybridization using said genomic DNA, cDNA, and probes.

23. A method of isolating a Nicotiana FucT DNA fragment comprising the steps of:
(a) identifying said Nicotiana FucT DNA fragment according to the method of claim 22; and
(b) isolating said Nicotiana FucT DNA fragment.

24. A method of identifying a Nicotiana FucT allele correlated with a low level of α-1,3-fucose residues on protein-bound N-glycans comprising the steps of:
(a) providing a population, optionally a mutagenized population, of different plant lines of a Nicotiana species or cultivar;
(b) identifying in each plant line of said population a Nicotiana FucT DNA fragment according to the method of claim 22;
(c) analyzing the level of α-1,3-fucose residues on protein-bound N-glycans of each plant line of said population and identifying those plant lines having a lower level of α-1,3-fucose residues on protein-bound N-glycans than other plant lines; and
(d) correlating the low level of α-1,3-fucose residues on protein-bound N-glycans in a plant line to the presence of a specific Nicotiana FucT allele.

25. A method of obtaining a Nicotiana plant cell or plant with a low level of β-1,2-xylose residues and core α-1,3-fucose residues on protein-bound N-glycans, comprising the steps of:
(a) identifying a Nicotiana XylT allele correlated with a low level of β-1,2-xylose residues on protein-bound N-glycans and introducing said Nicotiana XylT allele into a first plant of a Nicotiana plant line of choice;
(b) identifying a Nicotiana FucT allele correlated with a low level of α-1,3-fucose residues on protein-bound N-glycans according to the method of claim 24 and introducing said Nicotiana FucT allele into a second plant of a Nicotiana plant line of choice, wherein the plant line from which said second plant originates can be the same or not as the plant line from which said first plant originates;
(c) crossing a transformed plant obtained in step a) with a transformed plant obtained in step (b) to obtain transgenic Nicotiana plants; and
(d) optionally, identifying a transgenic Nicotiana plant which has a lower level of β-1,2-xylose residues and α-1,3-fucose residues on protein-bound N-glycans than an untransformed Nicotiana plant.

26. An isolated DNA fragment encoding a FucT protein of amino acid sequence SEQ ID NO.: 27 or an isolated DNA fragment comprising the nucleotide sequence of SEQ ID NO.: 26; or any part thereof comprising at least 20, at least 21, at least 22, at least 25, at least 50, at least 100, at least 150, or at least 200 contiguous nucleotides.

27. A chimeric gene comprising the following operably linked DNA fragments:
(a) a plant expressible promoter;
(b) a DNA region which, when transcribed, yields an RNA molecule capable of forming a double stranded RNA region by base-pairing at least between:
(i) an RNA region transcribed from a first DNA region comprising at least 18 out of 20-21, at least 19, at least 20, at least 21, at least 22, at least 25, at least 50, at least 100, at least 150, or at least 200, consecutive nucleotides of a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in antisense orientation;
(ii) an RNA region transcribed from a second DNA region comprising at least 18 out of 20-21, at least 19, at least 20, at least 21, at least 22, at least 25, at least 50, at least 100, at least 150, or at least 200, consecutive nucleotides of a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in sense orientation; and
(c) a DNA region comprising a transcription termination and polyadenylation signal functional in plants.

28. A chimeric gene comprising the following operably linked DNA fragments:
(a) a plant expressible promoter;
(b) a DNA region comprising at least 18 out of 20-21, at least 19, at least 20, at least 21, at least 22, at least 25, at least 50, at least 100, at least 150, or at least 200, consecutive nucleotides of a nucleotide sequence encoding a Nicotiana FucT protein of SEQ ID NO.: 27, or the complement thereof, or the nucleotide sequence of a Nicotiana FucT gene or a Nicotiana FucT cDNA of SEQ ID NO.: 26, or the complement thereof, in sense or antisense orientation; and
(c) a DNA region comprising a transcription termination and polyadenylation signal functional in plants.

29. A plant cell comprising:
(a) a first chimeric gene capable of producing a silencing RNA molecule, comprising a double stranded RNA (“dsRNA”) molecule, wherein the complementary RNA strands of such a dsRNA molecule comprises a part of a nucleotide sequence encoding a XyIT protein; and
(b) a second chimeric gene capable of producing a silencing RNA molecule, comprising a double stranded RNA (“dsRNA”) molecule, wherein the complementary RNA strands of such a dsRNA molecule comprises a part of a nucleotide sequence encoding a FucT protein; wherein said first and second chimeric genes are placed at unlinked positions in the genome of said plant cell.

30. The plant cell of claim 29, wherein said second chimeric gene comprises the following operably linked DNA fragments:
(a) a plant expressible promoter;
(b) a DNA region which, when transcribed, yields an RNA molecule capable of forming a double stranded RNA region by base-pairing at least between:
(i) an RNA region transcribed from a first DNA region comprising at least 18 out of 20-21, at least 19, at least 20, at least 21, at least 22, at least 25, at least 50, at least 100, at least 150, or at least 200, consecutive nucleotides of a nucleotide sequence encoding a Nicotiana FucT protein of SEQ ID NO.: 27, or the complement thereof, or selected from the nucleotide sequence of a Nicotiana FucT gene or a Nicotiana FucT cDNA of SEQ ID NO.: 26, or the complement thereof, in antisense orientation;
(ii) an RNA region transcribed from a second DNA region comprising at least 18 out of 20-21, at least 19, at least 20, at least 21, at least 22, at least 25, at least 50, at least 100, at least 150, or at least 200, consecutive nucleotides of a nucleotide sequence encoding a Nicotiana FucT protein of SEQ ID NO.: 27, or the complement thereof, or the nucleotide sequence of a Nicotiana FucT gene or a Nicotiana FucT cDNA of SEQ ID NO.: 26, or the complement thereof, in sense orientation; and
(c) a DNA region comprising a transcription termination and polyadenylation signal functional in plants.

31. The plant cell of claim 29, belonging to a Nicotiana species or cultivar.

32. The plant consisting essentially of the Nicotiana plant cell of claim 30.

33. A seed of the plant of claim 32.

34-38. (canceled)

39. The plant cell of claim 29, wherein said second chimeric gene comprises the following operably linked DNA fragments:
(a) a plant expressible promoter;
(b) a DNA region comprising at least 18 out of 20-21, at least 19, at least 20, at least 21, at least 22, at least 25, at least 50, at least 100, at least 150, or at least 200, consecutive nucleotides comprising a nucleotide sequence encoding a Nicotiana FucT protein of SEQ ID NO.: 27, or the complement thereof, or the nucleotide sequence of a Nicotiana FucT gene or a Nicotiana FucT cDNA of SEQ ID NO.: 26, or the complement thereof, in sense or antisense orientation; and
(c) a DNA region comprising a transcription termination and polyadenylation signal functional in plants.

40. The method of claim 6, wherein said FucT protein comprises a nucleotide sequence encoding the amino acid sequence of SEQ ID NO.: 27.

41. The method of claim 6, wherein said nucleotide sequence of FucT gene or a FucT cDNA comprises the sequence of SEQ ID NO.: 26.

42. The method of claim 21, wherein the Nicotiana species or cultivar is a Nicotiana bethamiana plant cell or plant.

43. The plant cell of claim 31, wherein the Nicotiana species or cultivar is Nicotiana benthamiana.
